(19) United States
(12) Patent Application Publication
(10) Pub. No.: US 2021/0089946 A1
(43) Pub. Date: Mar. 25, 2021

(54) VAPOR PREDICTION MODEL FOR A VAPORIZER DEVICE

(71) Applicant: JUUL Labs, Inc., San Francisco, CA (US)

(72) Inventors: Mika Pegors, San Francisco, CA (US); Arya Goudarzi, San Francisco, CA (US); Alexander Kudlick, San Francisco, CA (US); Conrad Legendy, Denver, CO (US)

(21) Appl. No.: 17/031,806

(22) Filed: Sep. 24, 2020

Related U.S. Application Data
(60) Provisional application No. 62/905,875, filed on Sep. 25, 2019.
(60) Provisional application No. 62/991,037, filed on Mar. 17, 2020.
(60) Provisional application No. 63/058,336, filed on Jul. 29, 2020.

(51) Int. Cl. G06N 5/04; A24F 40/50; G06N 20/00; G16H 20/17; G16H 40/67; G16H 40/40

(52) U.S. Cl. CPC G06N 5/04; A24F 40/50; G06N 20/00; G06Q 50/01; G16H 40/67; G16H 40/40; G16H 20/17

(57) ABSTRACT

Features relating to a model that predicts an amount of vapor production of a vaporizable material from a vaporizer device are provided. The model, for example a machine learning model and/or a statistical model, is developed as a function of various factors relating to operation of the vaporizer device. The model may be integrated in a system to provide user control and visibility into the amount of vapor production. In accordance with implementations of the current subject matter, an amount of consumed vapor may be determined. Such determination advantageously allows for a user to be informed of an amount consumed as well as optionally limit or otherwise control an amount consumed.

### CROSS-REFERENCE TO RELATED APPLICATIONS

This application claims priority to U.S. Provisional Patent Application No. 62/905,875, filed on Sep. 25, 2019, U.S. Provisional Patent Application No. 62/991,037, filed on Mar. 17, 2020, and U.S. Provisional Patent Application No. 63/058,336, filed on Jul. 29, 2020, the contents of which are herein incorporated by reference in their entirety.

### TECHNICAL FIELD

The current subject matter described herein relates generally to vaporizer devices, such as portable, personal vaporizer devices for generating and delivering an inhalable aerosol from one or more vaporizable materials, and more particularly relates to vaporizer devices configured to allow for control of an amount of vapor.

### BACKGROUND

Vaporizing devices, including electronic vaporizers or e-vaporizer devices, allow the delivery of vapor and aerosol containing one or more active ingredients by inhalation of the vapor and aerosol. Electronic vaporizer devices are gaining increasing popularity both for prescriptive medical use, in delivering medicaments, and for consumption of nicotine, tobacco, other liquid-based substances, and other plant-based smokeable materials, such as cannabis, including solid (e.g., loose-leaf or flower) materials, solid/liquid (e.g., suspensions, liquid-coated) materials, wax extracts, and prefilled pods (cartridges, wrapped containers, etc.) of such materials. Electronic vaporizer devices in particular may be portable, self-contained, and convenient for use.

### SUMMARY

Aspects of the current subject matter relate to predicting and/or determining an amount of vapor production of a vaporizable material by a vaporizer device. Aspects of the current subject matter also relate to controlling an amount of one or more vaporizable materials being vaporized by a vaporizer device.

According to an aspect of the current subject matter, a system includes at least one data processor and at least one memory storing instructions which, when executed by the at least one data processor, cause operations including receiving, at a trained model, at least one metric of a vaporizer device during a user puff on the vaporizer device; and processing, at the trained model, the at least one metric to predict an amount of vapor production of a vaporizable material from the vaporizer device during the user puff on the vaporizer device.

According to an inter-related aspect, a method includes receiving, at a trained model, at least one metric of a vaporizer device during a user puff on the vaporizer device; and processing, at the trained model, the at least one metric to predict an amount of vapor production of a vaporizable material from the vaporizer device during the user puff on the vaporizer device.

According to an inter-related aspect, a non-transitory computer readable medium is provided, the non-transitory computer readable medium storing instructions, which when executed by at least one data processor, result in operations including receiving, at a trained model, at least one metric of a vaporizer device during a user puff on the vaporizer device; and processing, at the trained model, the at least one metric to predict an amount of vapor production of a vaporizable material from the vaporizer device during the user puff on the vaporizer device.

In some variations, one or more of the features disclosed herein including the following features can optionally be included in any feasible combination. A model may be trained to provide the trained model configured to predict the amount of vapor production of the vaporizable material from the vaporizer device during the user puff on the vaporizer device. The training of the model may include processing, with the model, training data, the training data including a plurality of metrics corresponding to operation of the vaporizer device. The training data may include a plurality of values from a plurality of puffs on the vaporizer device. The model may include a machine learning model, a statistical model, and/or an analytical model. The model may include one of a regression model, a random forest model, an extra trees regression model, an adaptive boosted random forest regression model, an adaptive boosted extra trees regression model, a gradient boosting regression model, a support vector regression, a linear regression model, a ridge regression model, an elastic net regression model, and a lasso regression model, or a k-nearest neighbors regression model. The model may include the regression model, and the regression model may average predictions of regression trees, the regression trees individually built on a random subset of the training data and a random subset of variables. The at least one metric may include one or more of the following: a metric obtained during operation of the vaporizer device, an amount of energy used during the user puff, a temperature of a heating element of the vaporizer device, a differential pressure value of the vaporizer device, a resistance value of the heating element of the vaporizer device, a battery voltage of a battery of the vaporizer device, an amount of time to reach a setpoint temperature of the vaporizer device, an ambient pressure value, an amount of time of the user puff, an amount of energy to reach the setpoint temperature, an air path pressure in an air path of the vaporizer device, an ambient temperature, and an air path temperature in the air path of the vaporizer device. Based on the predicted amount of vapor production and an initial, total amount of vapor associated with a cartridge of the vaporizer device, a remaining amount of vapor may be determined; and upon a determination that the remaining amount of vapor is less than a predefined threshold, a notification to a user device associated with the vaporizer device may be provided. At a database, the predicted amount of vapor production may be stored with a cartridge identifier associated with the cartridge and a user identifier associated with a user. An indication of a desired amount of vapor production may be received; a determination may be made that the desired amount of vapor production equals or exceeds the predicted amount of vapor production; and, in response to the determination that the desired amount of vapor production equals or exceeds the predicted amount of vapor production, the vaporizer device may be caused to stop producing vapor. The predicted amount of vapor production may be summed over a plurality of user puffs.

According to an aspect of the current subject matter, a system includes at least one data processor and at least one memory storing instructions which, when executed by the at least one data processor, cause operations including receiving an indication of a selected dose amount of a vaporizable material for consumption by a user of a vaporizer device; providing the selected dose amount to a trained model to determine one or more settings to be applied by the vaporizer device; and applying the one or more settings to the vaporizer device to achieve the selected dose amount.

According to an inter-related aspect, a method includes receiving an indication of a selected dose amount of a vaporizable material for consumption by a user of a vaporizer device; providing the selected dose amount to a trained model to determine one or more settings to be applied by the vaporizer device; and applying the one or more settings to the vaporizer device to achieve the selected dose amount.

According to an inter-related aspect, a non-transitory computer readable medium is provided, the non-transitory computer readable medium storing instructions, which when executed by at least one data processor, result in operations including receiving an indication of a selected dose amount of a vaporizable material for consumption by a user of a vaporizer device; providing the selected dose amount to a trained model to determine one or more settings to be applied by the vaporizer device; and applying the one or more settings to the vaporizer device to achieve the selected dose amount.

In some variations, one or more of the features disclosed herein including the following features can optionally be included in any feasible combination. The selected dose amount may correlate with an amount of total particulate matter of the vaporizable material. The one or more settings may include an amount of energy to apply to a heating element of the vaporizer device. A temperature selection may be received; and the temperature selection with the selected dose amount may be provided to the trained model to determine the one or more settings to be applied by the vaporizer device. The trained model is trained to predict an amount of vapor production of the vaporizable material from the vaporizer device during a puff on the vaporizer device. The trained model is trained based at least on training data, the training data including a plurality of metrics corresponding to operation of the vaporizer device. The trained model may include a machine learning model, a statistical model, and/or an analytical model.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A-FIG. 1F illustrate features of a vaporizer device including a vaporizer body and a cartridge consistent with implementations of the current subject matter;

FIG. 2 is a schematic block diagram illustrating features of a vaporizer device having a cartridge and a vaporizer body consistent with implementations of the current subject matter;

FIG. 3 illustrates communication between a vaporizer device, a user device, and a server consistent with implementations of the current subject matter;

FIG. 4 is a schematic block diagram illustrating features of a vaporizer device consistent with implementations of the current subject matter;

FIG. 5A is a diagram depicting a training phase of a model consistent with implementations of the current subject matter;

FIG. 5B-5C are diagrams depicting use of a trained model consistent with implementations of the current subject matter;

FIG. 6A and FIG. 6B are diagrams depicting feature importance of a model consistent with implementations of the current subject matter;

FIGS. 7A-7C, 8A-8B, 9A-9B, and 10A-10D are diagrams illustrating features of exploratory data analysis for a model consistent with implementations of the current subject matter;

FIG. 11 illustrates residual plots depicting regression diagnostics consistent with implementations of the current subject matter;

FIG. 12 is an example of a chart for a process consistent with implementations of the current subject matter;

FIG. 13A and FIG. 13B are swim lane diagrams illustrating aspects of data communication consistent with implementations of the current subject matter;

FIG. 14 is another example of a chart for a process consistent with implementations of the current subject matter;

FIG. 15 is a block diagram illustrating aspects of utilizing a trained model consistent with implementations of the current subject matter;

FIG. 16 is another example of a chart for a process consistent with some implementations of the current subject matter; and

FIG. 17 is yet another example of a chart for a process consistent with some implementations of the current subject matter.

When practical, similar reference numbers denote similar structures, features, or elements.

### DETAILED DESCRIPTION

Aspects of the current subject matter relate to a model (herein also referred to as an analytical model, a machine learning model, and/or a statistical model) that predicts vapor production of a vaporizable material by a vaporizer device, the vapor production being a function of various metrics relating to operation of the vaporizer device (e.g., sensor data and other parameters). Aspects of the current subject matter also relate to integration of the model into one or more systems to provide user control and visibility into the vapor production. For example, the model may be integrated into a connected device system to provide user control and visibility into the vapor production via one or more connected devices. Additional aspects of the current subject matter relate to utilizing the model to provide a standardized amount of vapor.

In accordance with some implementations of the current subject matter, an amount of produced vapor may be determined. Such determination advantageously allows for a user to be informed of an amount vaporized and/or consumed as well as optionally limit or otherwise control an amount vaporized and/or consumed.

For example, a dose control implementation consistent with implementations of the current subject matter provides for monitoring an amount of vapor produced against a desired or predefined dose or amount of vapor. When the amount of vapor produced equals or exceeds the desired or predefined dose, an indication may be sent to a user and/or the vaporizer device may cease operation for a period of time.

As another example, a fuel gauge implementation consistent with implementations of the current subject matter provides for monitoring an amount of vapor produced and providing to a user an indication of a remaining amount of vapor.

In accordance with some implementations of the current subject matter, a desired amount of vapor for production may be indicated or otherwise selected (e.g., user selected). The model consistent with implementations of the current subject matter may be used to determine when the desired amount of vapor is produced by the vaporizer device, which may be used to affect operation of the vaporizer device. The model consistent with implementations of the current subject matter may also be used to determine one or more settings associated with the vaporizer device to achieve the desired amount of vapor. The model consistent with implementations of the current subject matter may also be used to provide usage data related to use of the vaporizer device.

Additional aspects of the current subject matter relate to determining an amount of vapor (also referred to herein as an inhalable aerosol) consumed by a user, as well as providing a predetermined or preselected amount of vapor for consumption by a user. In accordance with implementations of the current subject matter, by obtaining, identifying, and/or determining characteristics of a vaporizable material, characteristics of the vaporizer device including, for example, a cartridge that delivers the vapor, and/or a user's behavior, an amount of the consumed vapor may be determined. Such determination advantageously allows for a user to be informed of an amount consumed as well as optionally limit or otherwise control an amount consumed.

Before providing additional details regarding aspects of the prediction of vapor production of a vaporizable material by a vaporizer device, the following provides a description of some examples of vaporizer devices in which aspects of the current subject matter may be implemented. The following descriptions are meant to be exemplary, and aspects related to the prediction of vapor production by a vaporizer device consistent with the current subject matter are not limited to the example vaporizer devices described herein.

Implementations of the current subject matter include devices relating to vaporizing of one or more materials for inhalation by a user. The term “vaporizer” may be used generically in the following description and may refer to a vaporizer device, such as an electronic vaporizer. Vaporizers consistent with the current subject matter may be referred to by various terms such as inhalable aerosol devices, aerosolizers, vaporization devices, electronic vaping devices, electronic vaporizers, vape pens, etc. Examples of vaporizers consistent with implementations of the current subject matter include electronic vaporizers, electronic cigarettes, e-cigarettes, or the like. In general, such vaporizers are often portable, hand-held devices that heat a vaporizable material to provide an inhalable dose of the material. The vaporizer may include a heater configured to heat a vaporizable material which results in the production of one or more gas-phase components of the vaporizable material. A vaporizable material may include liquid and/or oil-type plant materials, or a semi-solid like a wax, or plant material such as leaves or flowers, either raw or processed. The gas-phase components of the vaporizable material may condense after being vaporized such that an aerosol is formed in a flowing air stream that is deliverable for inhalation by a user. The vaporizers may, in some implementations of the current subject matter, be particularly adapted for use with an oil-based vaporizable material, such as cannabis-derived oils although other types of vaporizable materials may be used as well.

One or more features of the current subject matter, including one or more of a cartridge (also referred to as a vaporizer cartridge or pod) and a reusable vaporizer device body (also referred to as a vaporizer device base, a body, a vaporizer body, or a base), may be employed with a suitable vaporizable material (where suitable refers in this context to being usable with a device whose properties, settings, etc. are configured or configurable to be compatible for use with the vaporizable material). The vaporizable material may include one or more liquids, such as oils, extracts, aqueous or other solutions, etc., of one or more substances that may be desirably provided in the form of an inhalable aerosol. The cartridge may be inserted into the vaporizer body, and then the vaporizable material heated which results in the inhalable aerosol.

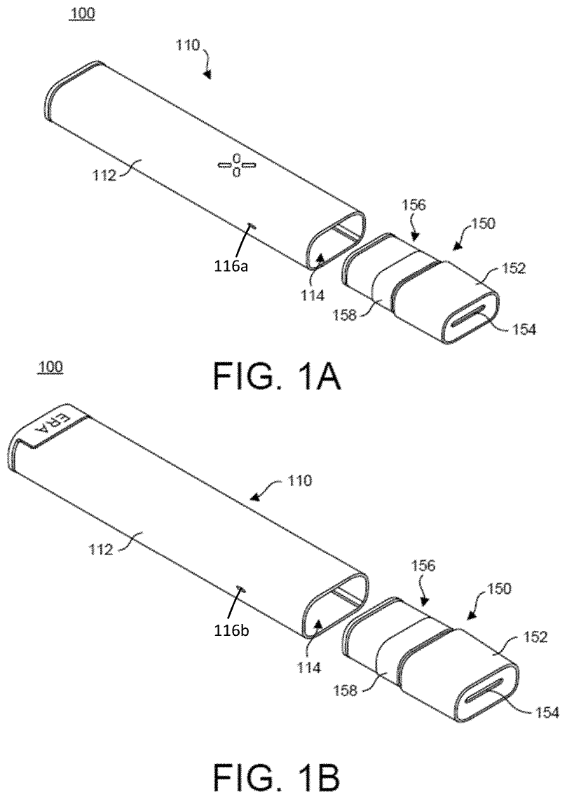

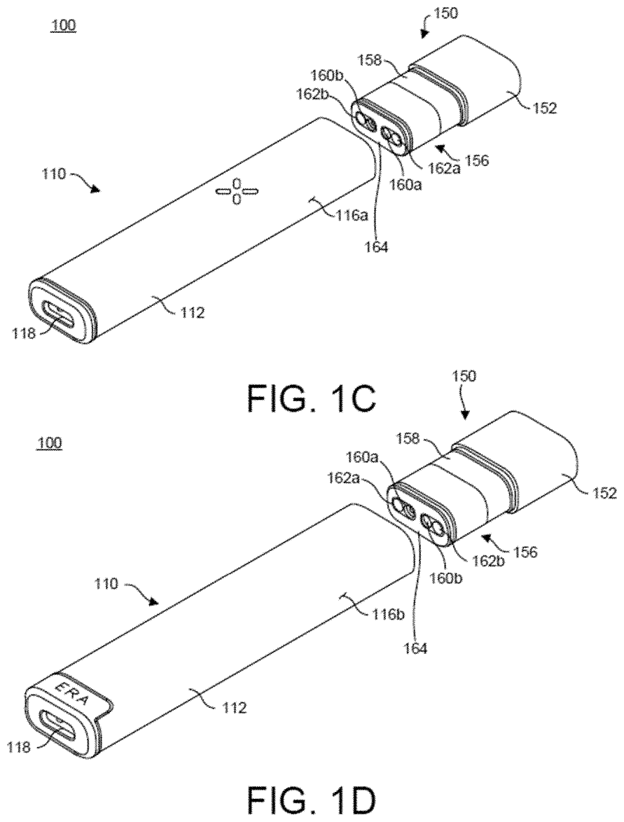

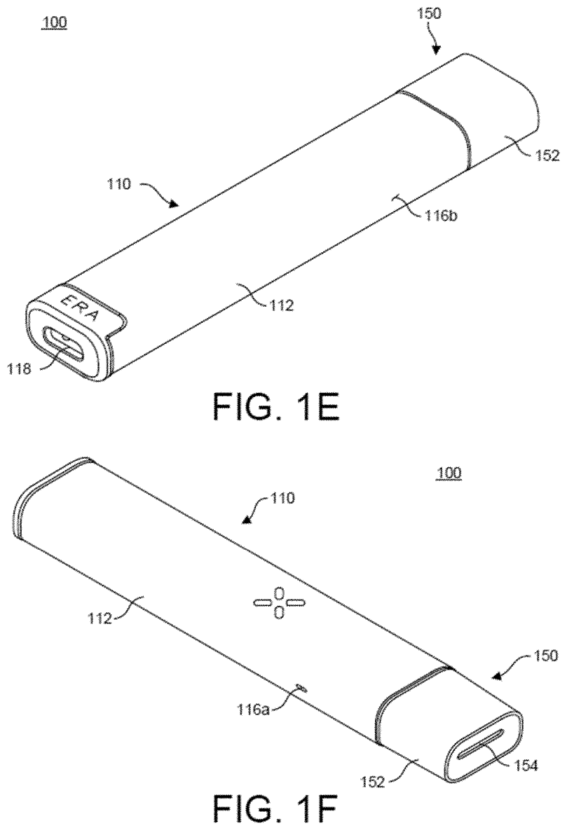

FIG. 1A-FIG. 1F illustrates features of a vaporizer device 100 including a vaporizer body 110 and a cartridge 150 consistent with implementations of the current subject matter. FIG. 1A is a bottom perspective view, and FIG. 1B is a top perspective view of the vaporizer device 100 with the cartridge 150 separated from a cartridge receptacle 114 on the vaporizer body 110. Both of the views in FIG. 1A and FIG. 1B are shown looking towards a mouthpiece 152 of the cartridge 150. FIG. 1C is a bottom perspective view, and FIG. 1D is a top perspective view of the vaporizer device with the cartridge 150 separated from the cartridge receptacle 114 of the vaporizer body 110. FIG. 1C and FIG. 1D are shown looking toward the distal end of the vaporizer body 110. FIG. 1E is top perspective view, and FIG. 1F is a bottom perspective view of the vaporizer device 100 with the cartridge 150 engaged for use with the vaporizer body 110.

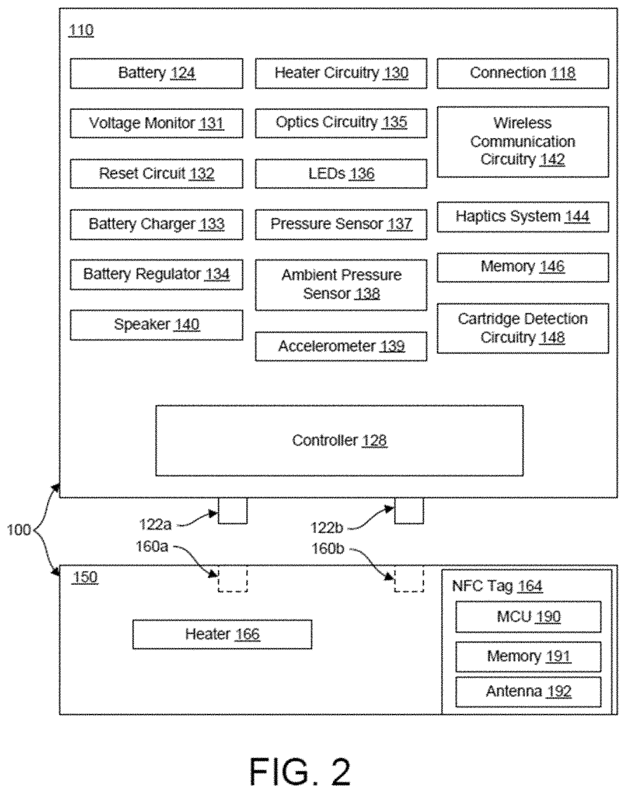

As shown in FIG. 1A-FIG. 1D, the cartridge 150 includes, at the proximal end, a mouthpiece 152 that is attached over a cartridge body 156 that forms a reservoir or tank 158 that holds a vaporizable material. The cartridge body 156 may be transparent, translucent, opaque, or a combination thereof. The mouthpiece 152 may include one or more openings 154 (see FIG. 1A, FIG. 1B, FIG. 1F) at the proximal end out of which vapor may be inhaled, by drawing breath through the vaporizer device 100. The distal end of the cartridge body 156 may couple to and be secured to the vaporizer body 110 within the cartridge receptacle 114 of the vaporizer body 110. Power pin receptacles 160a,b (see FIG. 1C, FIG. 1D) of the cartridge 150 mate with respective power pins or contacts 122a,b (see, for example, FIG. 2) of the vaporizer body 110 that extend into the cartridge receptacle 114. The cartridge 150 also includes air flow inlets 162a,b on the distal end of the cartridge body 156.

A tag 164, such as a data tag, a near-field communication (NFC) tag, or other type of wireless transceiver or communication tag, may be positioned on at least a portion of the distal end of the cartridge body 156. As shown in FIG. 1C and FIG. 1D, the tag 164 may substantially surround the power pin receptacles 160a,b and the air flow inlets 162a,b, although other configurations of the tag 164 may be implemented as well. For example, the tag 164 may be positioned between the power pin receptacle 160a and the power pin receptacle 160b, or the tag 164 may be shaped as a circle, partial circle, oval, partial oval, or any polygonal shape encircling or partially encircling the power pin receptacles 160a,b and the air flow inlets 162a,b or a portion thereof.

In the example of FIG. 1A, the vaporizer body 110 has an outer shell or cover 112 that may be made of various types of materials, including for example aluminum (e.g., AL6063), stainless steel, glass, ceramic, titanium, plastic (e.g., Acrylonitrile Butadiene Styrene (ABS), Nylon, Polycarbonate (PC), Polyethersulfone (PESU), and the like), fiberglass, carbon fiber, and any hard, durable material. The proximal end of the vaporizer body 110 includes an opening forming the cartridge receptacle 114, and the distal end of the vaporizer body 110 includes a connection 118, such as, for example, a universal serial bus Type C (USB-C) connection and/or the like. The cartridge receptacle 114 portion of the vaporizer body 110 includes one or more openings (air inlets) 116a,b that extend through the outer shell 112 to allow airflow therein, as described in more detail below. The vaporizer body 110 as shown has an elongated, flattened tubular shape that is curvature-continuous, although the vaporizer body 110 is not limited to such a shape. The vaporizer body 110 may take the form of other shapes, such as, for example, a rectangular box, a cylinder, and the like.

The cartridge 150 may fit within the cartridge receptacle 114 by a friction fit, snap fit, and/or other types of secure connection. The cartridge 150 may have a rim, ridge, protrusion, and/or the like for engaging a complimentary portion of the vaporizer body 110. While fitted within the cartridge receptacle 114, the cartridge 150 may be held securely within but still allow for being easily withdrawn to remove the cartridge 150.

Although FIG. 1A-FIG. 1F illustrate a certain configuration of the vaporizer device 100, the vaporizer device 100 may take other configurations as well.

FIG. 2 is a schematic block diagram illustrating components of the vaporizer device 100 having the cartridge 150 and the vaporizer body 110 consistent with implementations of the current subject matter. Included in the vaporizer body 110 is a controller 128 that includes at least one processor and/or at least one memory configured to control and manage various operations among the components of the vaporizer device 100 described herein.

Heater control circuitry 130 of the vaporizer body 110 controls a heater 166 of the cartridge 150. The heater 166 may generate heat to provide vaporization of the vaporizable material. For example, the heater 166 may include a heating coil (e.g., a resistive heater) in thermal contact with a wick which absorbs the vaporizable material, as described in further detail below.

A battery 124 is included in the vaporizer body 110, and the controller 128 may control and/or communicate with a voltage monitor 131 which includes circuitry configured to monitor the battery voltage, a reset circuit 132 configured to reset (e.g., shut down the vaporizer device 100 and/or restart the vaporizer device 100 in a certain state), a battery charger 133, and a battery regulator 134 (which may regulate the battery output, regulate charging/discharging of the battery, and provide alerts to indicate when the battery charge is low, etc.).

The power pins 122a,b of the vaporizer body 110 engage the complementary power pin receptacles 160a,b of the cartridge 150 when the cartridge 150 is engaged with the vaporizer body 110. Alternatively, power pins may be part of the cartridge 150 for engaging complementary power pin receptacles of the vaporizer body 110. The engagement allows for the transfer of energy from an internal power source (e.g., the battery 124) to the heater 166 in the cartridge 150. The controller 128 may regulate the power flow (e.g., an amount or current and/or a voltage amount) to control a temperature at which the heater 166 heats the vaporizable material contained in the reservoir 158. According to implementations of the current subject matter, a variety of electrical connectors other than a pogo-pin and complementary pin receptacle configuration may be used to electrically connect the vaporizer body 110 and the cartridge 150, such as for example, a plug and socket connector.

The controller 128 may control and/or communicate with optics circuitry 135 (which controls and/or communicates with one or more displays such as LEDs 136 which may provide user interface output indications), a pressure sensor 137, an ambient pressure sensor 138, an accelerometer 139, and/or a speaker 140 configured to generate sound or other feedback to a user.

The pressure sensor 137 may be configured to sense a user drawing (i.e., inhaling) on the mouthpiece 152 and activate the heater control circuitry 130 of the vaporizer body 110 to accordingly control the heater 166 of the cartridge 150. In this way, the amount of current supplied to the heater 166 may be varied according the user's draw (e.g., additional current may be supplied during a draw, but reduced when there is not a draw taking place). The ambient pressure sensor 138 may be included for atmospheric reference to reduce sensitivity to ambient pressure changes and may be utilized to reduce false positives potentially detected by the pressure sensor 137 when measuring draws from the mouthpiece 152.

The accelerometer 139 (and/or other motion sensors, capacitive sensors, flow sensors, strain gauge(s), or the like) may be used to detect user handling and interaction, for example, to detect movement of the vaporizer body 110 (such as, for example, tapping, rolling, and/or any other deliberate movement associated with the vaporizer body 110).

The vaporizer body 110, as shown in FIG. 2, includes wireless communication circuitry 142 that is connected to and/or controlled by the controller 128. The wireless communication circuitry 142 may include a near-field communication (NFC) antenna that is configured to read from and/or write to the tag 164 of the cartridge 150. Alternatively or additionally, the wireless communication circuitry 142 may be configured to automatically detect the cartridge 150 as it is being inserted into the vaporizer body 110. In some implementations, data exchanges between the vaporizer body 110 and the cartridge 150 take place over NFC. In some implementations, data exchanges between the vaporizer body 110 and the cartridge 150 may take place via a wired connection such as various wired data protocols.

The wireless communication circuitry 142 may include additional components including circuitry for other communication technology modes, such as Bluetooth circuitry, Bluetooth Low Energy circuitry, Wi-Fi circuitry, cellular (e.g., LTE, 4G, and/or 5G) circuitry, and associated circuitry (e.g., control circuitry), for communication with other devices. For example, the vaporizer body 110 may be configured to wirelessly communicate with a remote processor (e.g., a smartphone, a tablet, a computer, wearable electronics, a cloud server, and/or processor based devices) through the wireless communication circuitry 142, and the vaporizer body 110 may through this communication receive information including control information (e.g., for setting temperature, resetting a dose counter, etc.) from and/or transmit output information (e.g., dose information, operational information, error information, temperature setting information, charge/battery information, etc.) to one or more of the remote processors.

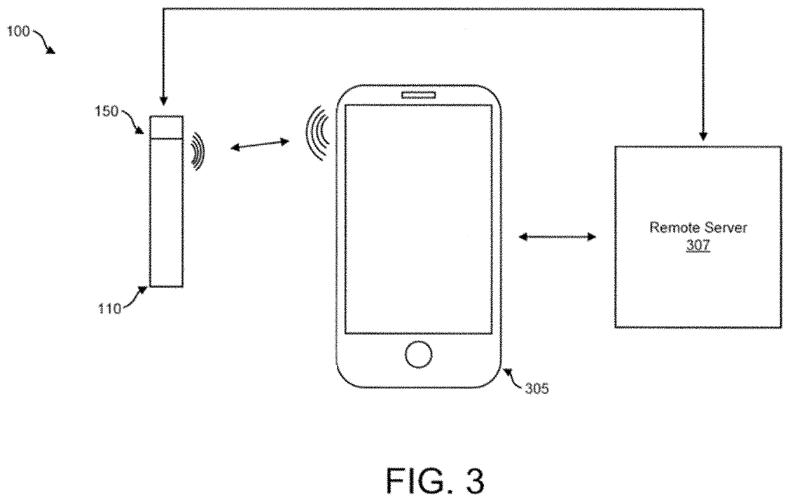

The tag 164 may be a type of wireless transceiver and may include a microcontroller unit (MCU) 190, a memory 191, and an antenna 192 (e.g., an NFC antenna) to perform the various functionalities described below with further reference to FIG. 3. NFC tag 164 may be, for example, a 1 Kbit or a 2 Kbit tag that is of type ISO/IEC 15693. NFC tags with other specifications may also be used. The tag 164 may be implemented as active NFC, enabling reading and/or writing information via NFC with other NFC compatible devices including a remote processor, another vaporizer device, and/or wireless communication circuitry 142. Alternatively, the tag 164 may be implemented using passive NFC technology, in which case other NFC compatible devices (e.g., a remote processor, another vaporizer device, and/or wireless communication circuitry 142) may only be able to read information from the tag 164.

The vaporizer body 110 may include a haptics system 144, such as an actuator, a linear resonant actuator (LRA), an eccentric rotating mass (ERM) motor, or the like that provide haptic feedback such as a vibration as a “find my device” feature or as a control or other type of user feedback signal. For example, using an app running on a user device (such as, for example, a user device 305 shown in FIG. 3), a user may indicate that he/she cannot locate his/her vaporizer device 100. Through communication via the wireless communication circuitry 142, the controller 128 sends a signal to the haptics system 144, instructing the haptics system 144 to provide haptic feedback (e.g., a vibration). The controller 128 may additionally or alternatively provide a signal to the speaker 140 to emit a sound or series of sounds. The haptics system 144 and/or speaker 140 may also provide control and usage feedback to the user of the vaporizer device 100; for example, providing haptic and/or audio feedback when a particular amount of a vaporizable material has been used or when a period of time since last use has elapsed. Alternatively or additionally, haptic and/or audio feedback may be provided as a user cycles through various settings of the vaporizer device 100. Alternatively or additionally, the haptics system 144 and/or speaker 140 may signal when a certain amount of battery power is left (e.g., a low battery warning and recharge needed warning) and/or when a certain amount of vaporizable material remains (e.g., a low vaporizable material warning and/or time to replace the cartridge 150). Alternatively or additionally, the haptics system 144 and/or speaker 140 may also provide usage feedback and/or control of the configuration of the vaporizer device 100 (e.g., allowing the change of a configuration, such as target heating rate, heating rate, etc.).

The vaporizer body 110 may include circuitry for sensing/detecting when a cartridge 150 is connected and/or removed from the vaporizer body 110. For example, cartridge-detection circuitry 148 may determine when the cartridge 150 is connected to the vaporizer body 110 based on an electrical state of the power pins 122a,b within the cartridge receptacle 114. For example, when the cartridge 150 is present, there may be a certain voltage, current, and/or resistance associated with the power pins 122a,b, when compared to when the cartridge 150 is not present. Alternatively or additionally, the tag 164 may also be used to detect when the cartridge 150 is connected to the vaporizer body 110.

The vaporizer body 110 also includes the connection (e.g., USB-C connection, micro-USB connection, and/or other types of connectors) 118 for coupling the vaporizer body 110 to a charger to enable charging the internal battery 124. Alternatively or additionally, electrical inductive charging (also referred to as wireless charging) may be used, in which case the vaporizer body 110 would include inductive charging circuitry to enable charging. The connection 118 at FIG. 2 may also be used for a data connection between a computing device and the controller 128, which may facilitate development activities such as, for example, programming and debugging, for example.

The vaporizer body 110 may also include a memory 146 that is part of the controller 128 or is in communication with the controller 128. The memory 146 may include volatile and/or non-volatile memory or provide data storage. In some implementations, the memory 146 may include 8 Mbit of flash memory, although the memory is not limited to this and other types of memory may be implemented as well.

FIG. 3 illustrates communication between the vaporizer device 100 (including the vaporizer body 110 and the cartridge 150), the user device 305 (e.g., a smartphone, tablet, laptop, desktop computer, a workstation, and/or the like), and a remote server 307 (e.g., a server coupled to a network, a cloud server coupled to the Internet, and/or the like) consistent with implementations of the current subject matter. The user device 305 wirelessly communicates with the vaporizer device 100. A remote server 307 may communicate directly with the vaporizer device 100 or through the user device 305. The vaporizer body 110 may communicate with the user device 305 and/or the remote server 307 through the wireless communication circuitry 142. In some implementations, the cartridge 150 may establish through the tag 164 communication with the vaporizer body 110, the user device 305, and/or the remote server 307. While the user device 305 in FIG. 3 is depicted as a type of handheld mobile device, the user device 305 consistent with implementations of the current subject matter is not so limited and may be, as indicated, various other types of user computing devices.

An application software (“app”) running on at least one of the remote processors (the user device 305 and/or the remote server 307) may be configured to control operational aspects of the vaporizer device 100 and receive information relating to operation of the vaporizer device 100. For example, the app may provide a user with capabilities to input or set desired properties or effects, such as, for example, a particular temperature or desired dose, which is then communicated to the controller 128 of the vaporizer body 110 through the wireless communication circuitry 142. The app may also provide a user with functionality to select one or more sets of suggested properties or effects that may be based on the particular type of vaporizable material in the cartridge 150. For example, the app may allow adjusting heating based on the type of vaporizable material, the user's (of the vaporizer device 100) preferences or desired experience, and/or the like. The app may be a mobile app and/or a browser-based or web app. For example, the functionality of the app may be accessible through one or more web browsers running on one or more types of user computing devices.

Data read from the tag 164 from the wireless communication circuitry 142 of the vaporizer body 110 may be transferred to one or more of the remote processors (e.g., the user device 305 and/or the remote server 307) to which it is connected, which allows for the app running on the one or more processors to access and utilize the read data for a variety of purposes. For example, the read data relating to the cartridge 150 may be used for providing recommended temperatures, dose control, usage tracking, and/or assembly information.

The cartridge 150 may also communicate directly, through the tag 164, with other devices. This enables data relating to the cartridge 150 to be written to/read from the tag 164, without interfacing with the vaporizer body 110. The tag 164 thus allows for identifying information (e.g., pod ID, batch ID, etc.) related to the cartridge 150 to be associated with the cartridge 150 by one or more remote processors. For example, when the cartridge 150 is filled with a certain type of vaporizable material, this information may be transmitted to the tag 164 by filling equipment. Then, the vaporizer body 110 is able to obtain this information from the tag 164 (e.g., via the wireless communication circuitry 142 at the vaporizer body 110) to identify the vaporizable material currently being used and accordingly adjust the controller 128 based on, for example, user-defined criteria or pre-set parameters associated with the particular type of vaporizable material (set by a manufacturer or as determined based upon user experiences/feedback aggregated from other users). For example, a user may establish (via the app) a set of criteria relating to desired effects for or usage of one or more types of vaporizable materials. When a certain vaporizable material is identified, based on communication via the tag 164, the controller 128 may accordingly adopt the established set of criteria, which may include, for example, temperature and dose, for that particular vaporizable material.

Consistent with implementations of the current subject matter, the vaporizable material used with the vaporizer device may be provided within the cartridge. The vaporizer device may be a cartridge-using vaporizer device, a cartridge-less vaporizer device, or a multi-use vaporizer device capable of use with or without a cartridge. For example, a multi-use vaporizer device may include a heating chamber (e.g., an oven) configured to receive the vaporizable material directly in the heating chamber and also configured to receive the cartridge having a reservoir or the like for holding the vaporizable material. In various implementations, the vaporizer device may be configured for use with liquid vaporizable material (e.g., a carrier solution in which an active and/or inactive ingredient(s) are suspended or held in solution or a liquid form of the vaporizable material itself) or solid vaporizable material. Solid vaporizable material may include a plant material that emits some part of the plant material as the vaporizable material (e.g., such that some part of the plant material remains as waste after the vaporizable material is emitted for inhalation by a user) or optionally may be a solid form of the vaporizable material itself such that all of the solid material may eventually be vaporized for inhalation. Liquid vaporizable material may likewise be capable of being completely vaporized or may include some part of the liquid material that remains after all of the material suitable for inhalation has been consumed.

As described above, the vaporizer device 100 and/or the user device 305 that is part of a vaporizer system as defined above may include a user interface (e.g., including an app or application software) that may be executed on the user device 305 in communication, which may be configured to determine, display, enforce, and/or meter dosing.

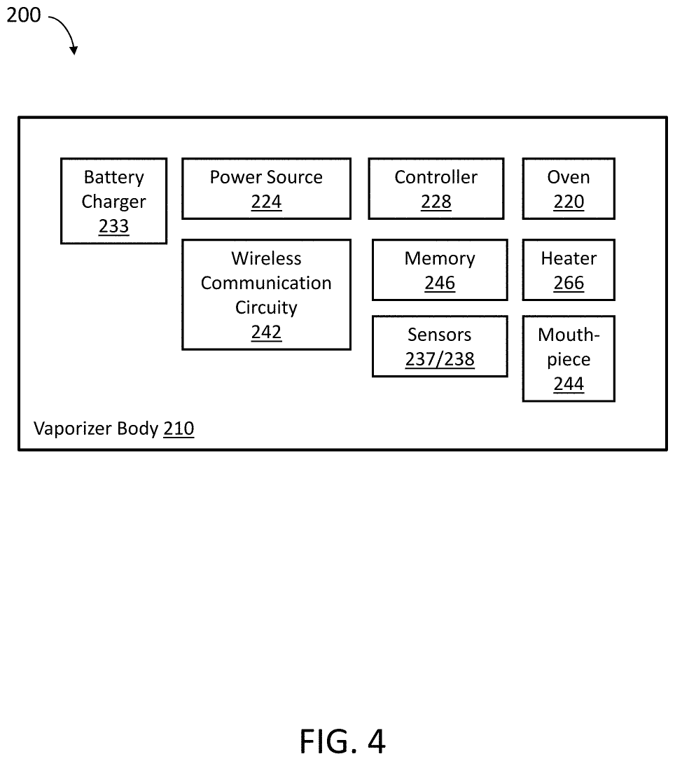

FIG. 4 is a schematic block diagram illustrating features of another vaporizer device 200 consistent with implementations of the current subject matter. The vaporizer device 200 does not require use of a cartridge (but may still optionally accept a cartridge), but may instead use a loose-leaf material. The vaporizer device 200 in FIG. 4 includes an oven 220 (e.g., vaporization chamber) in which loose vaporizable material may be placed. Many of the same elements present in the vaporizer device 100 using cartridge 150 shown in FIG. 1A-FIG. 1F and FIG. 2 may also be included as part of the vaporizer device 200. For example, the vaporizer device 200 may include a vaporizer body 210 with controller 228, wireless communication circuitry 242, and/or memory 246. A power source 224 (e.g., battery, capacitor, etc.) may be charged by a battery charger 233 (and may include charging control circuitry, not shown). The vaporizer device 200 may also include one or more sensors 237, 238. In addition, the vaporizer device 200 may include one or more heaters 266 that heat the oven 220. The heater 266 may be controlled using the resistance of the heater 266 to determine the temperature of the heater, e.g., by using the temperature coefficient of resistivity for the heater 266. Convection heating methods may be used. A mouthpiece 244 is also included.

The vaporizer device consistent with implementations of the current subject matter may be configured to facilitate social interaction through the vaporizer device. For example, the vaporizer device may be configured to share usage information with others, such as third parties including health care providers, etc., for better prescription and administration of medical treatment. The vaporizer device may also be configured to communicate with non-medical third parties (e.g., friends, colleagues, etc.), and with unknown third parties (making some or all information publically available). In some implementations, the vaporizer device described herein, either by itself or in communication with one or more communications devices that are part of a system, may identify and provide information about the operation, status, or user input from the vaporizer device to a public or private network.

Software, firmware, or hardware that is separate or separable from the vaporizer device and that wirelessly communicates with the vaporizer device may be provided as described with respect to FIG. 3. For example, applications (“apps”) may be executed on a processor of a portable and/or wearable device, including smartphones, smartwatches, and the like, which may be referred to as a personal digital device, a user device, or optionally just a device (e.g., user device 305 in FIG. 3) that is part of a connected system. These digital devices may provide an interface for the user to engage and interact with functions related to the vaporizer device, including communication of data to and from the vaporizer device to the digital device or the like and/or additional third party processor (e.g., servers such as the remote server 307 in FIG. 3). For example, a user may control some aspects of the vaporizer device (temperature, session size, etc.) and/or data transmission and data receiving to and from the vaporizer device, optionally over a wireless communication channel between first communication hardware of the digital device and second communication hardware of the vaporizer device. Data may be communicated in response to one or more actions of the user (e.g., including interactions with a user interface displayed on the device), and/or as a background operation such that the user does not have to initiate or authorize the data communication process.

User interfaces may be deployed on the digital device and may aid the user in operating the vaporizer device. For example, the user interface operating on the digital device may include icons and text elements that may inform the user of various ways that vaporizer settings may be adjusted or configured by the user. In this manner (or in others consistent with the current subject matter) information about the vaporizer device may be presented using a user interface displayed by the digital device. Icons and/or text elements may be provided to allow the user to see information regarding one or more statuses of the vaporizer device, such as battery information (charge remaining, draws remaining, time to charge, charging, etc.), cartridge status (e.g., type of cartridge and vaporizable material, fill status of cartridge, etc.), and other device statuses or information. Icons and/or text elements may be provided to allow the user to update internal software (a.k.a., firmware) in the vaporizer device. Icons and text elements may be provided to allow the user to set security and/or authorization features of the vaporizer device, such as setting a PIN code to activate the vaporizer device or the use of personal biometric information as a way of authentication. Icons and text elements may be provided to allow the user to configure foreground data sharing and related settings.

The vaporizer device may perform onboard data gathering, data analysis, and/or data transmission methods. As mentioned, the vaporizer device having wired or wireless communication capability may interface with digital consumer technology products such as smart phones, tablet computers, laptop/netbook/desktop computers, wearable wireless technologies such as “smart watches,” and other wearable technology such as Google “Glass,” or similar through the use of programming, software, firmware, GUI, wireless communication, wired communication, and/or software commonly referred to as application(s) or “apps.” A wired communication connection may be used to interface the vaporizer device to digital consumer technology products for the purpose of the transmission and exchange of data to/from the vaporizer device from/to the digital consumer technology products (and thereby also interfacing with apps running on the digital consumer technology products). A wireless communication connection may be used to interface the vaporizer device to digital consumer technology products for the transmission and exchange of data to/from the vaporizer device from/to the digital wireless interface. The vaporizer device may use a wireless interface that includes one or more of an infrared (IR) transmitter, a Bluetooth interface, an 802.11 specified interface, and/or communications with a cellular telephone network in order to communicate with consumer technology.

The vaporizer device and/or the user device as defined above may be used for any of one or more functions, such as controlling dosing (e.g., dose monitoring, dose setting, dose limiting, user tracking, etc.), controlling sessioning (e.g., session monitoring, session setting, session limiting, user tracking, etc.), obtaining locational information (e.g., location of other users, retailer/commercial venue locations, vaping locations, relative or absolute location of the vaporizer device itself, etc.), vaporizer device personalization (e.g., naming the vaporizer device, locking and/or password protecting the vaporizer device, adjusting one or more parental controls, associating the vaporizer device with a user group, registering the vaporizer device with a manufacturer or warranty maintenance organization, etc.), engaging in social activities (e.g., games, social media communications, interacting with one or more groups, etc.) with other users, or the like. Implementations of the current subject matter refer to doses of aerosol for consumption. According to aspects of the current subject matter, a dose is defined as a fixed amount of aerosol generated by the vaporizer device for consumption by the user as a number of puffs taken by the user until the fixed amount of aerosol is consumed or inhaled. In some implementations, a dose may also be referred to as a session; and the terms dose and session may be used interchangeably herein. In some instances, a dose is fixed or selected by an entity, such as a manufacturer or distributor of the cartridge or the vaporizer device, or a care giver, medical facility, and/or the like for controlling consumption by the user. In some instances, the dose is user adjustable and/or user configurable. In some instances, the dose is represented by a dose size and/or a dose size setting. The dose may be stored for transmission and/or display, as described herein. Further, in accordance with implementations of the current subject matter, the dose may be used to control operation of the vaporizer device and may be established or set by a user.

The vaporizer device and/or vaporizer system provided by the communication between the vaporizer device 100, the user device 305, and/or the remote server 307 (described with reference to FIG. 3) may include dose control and/or dose metering. In some aspects, a user may find it desirable to monitor and/or control consumption of the vaporizable material. Such monitoring and/or control may beneficially allow a user to adjust an amount of vaporizable material available over a certain time period or session, a total time allowed for using the vaporizer, a time period between vaporizer doses, other consumption settings to meet the needs of the user, and/or the like.

For example, in accordance with implementations of the current subject matter, various preset dose configurations may be established to control a dose for the user. These preset dose configurations may be displayed to the user on a user interface, allowing for the user to select a desired dose. The preset dose configurations may relate to dose size, where dose size refers to size or amount of vaporizable material. According to some aspects, available dose sizes may include micro, small, medium, or large, signifying how much vapor is being produced for inhalation by a user. Other descriptive terms or identifiers (e.g., symbols or the like) may also be used for dose sizes, and the implementations described herein are not limited to the specific terms describing the dose size.

Additionally, in accordance with implementations of the current subject matter, a temperature may be selected to be used for the dose. The temperature may be selected to adjust the strength of vapor being produced (for example, higher temperatures may produce a stronger vapor compared to vapor produced from a lower temperature).

The temperature may be based on a user selection via selection on the vaporizer application. For example, the temperature of the vaporizer device may be adjusted by using a graphical user interface that allows both gross and precise control of the vaporizer temperature with, for example, a single finger. For example, a display of the temperature visually indicating the current temperature and/or target temperature of the vaporizer may be used to adjust the temperature up or down (within a range).

Alternatively, a preset temperature (which may be user, system, or cartridge defined and may be based on various factors such as for example user preferences, crowdsourced information, type of vaporizable material, and/or the like) may be applied. If a user does not specify a desired temperature, the preset temperature may be a default setting (e.g., a default provided by the vaporizer device or a default corresponding the vaporizable material (e.g., a strain of cannabinoids)). In other examples, the user selection may override the preset temperature. In accordance with some implementations of the current subject matter, once a dose starts, temperature is set and cannot be changed. In accordance with additional implementations, a temperature setting may carry over from a previous use.

Preset temperature settings may be chosen based on desired outcomes. For example, one such setting may initiate a temperature boost when puffing and an auto cool down when not puffing; while another setting may gradually ramp up the temperature. Some preset temperature settings may aggressively affect temperature and thus vapor production more than other preset temperature settings.

Once the dose size and/or temperature are selected, through for example user interaction on the vaporizer application, use of the vaporizer device according to the selected dose size may begin. Alternatively, operation of the vaporizer device may begin once other feedback or input is received, such as for example a user inhalation or puff or other defined action.

A visual indication of the status of the vaporizer dose may be provided to the user via the vaporizer application. For example, a status or progress bar indicating a completed percentage of the vaporizer dose may be displayed and continuously updated (i.e., progress is updated live while puffing). The progress bar may be indicative of the amount of power supplied to a heating element and may be a representation indicating amount of vaporizable material inhaled.

According to some aspects, once the vaporizer dose is completed, the vaporizer may be locked for a preset amount of time, for example 30 seconds. Other time periods may be used for the locked amount, and such time periods may be user or system defined. During such a lockout period, user puffing does not produce any vapor. Once the lockout period ends, the dose size may be set as the same as the previous dose unless otherwise updated by the user. There may be an option to overrule or end the lockout period.

In accordance with implementations of the current subject matter, incorporating dose options with temperature control provides for an extra level of user control. Additionally, the session size and temperature selection options allow for a user to control and replicate experiences (e.g., by using settings from previous sessions).

In general, any of the vaporizer devices described herein may estimate, measure, and/or predict the amount of vapor and/or material (including active ingredients) in the vapor that may be delivered to a user. For example, the devices described herein may be used to determine and/or control dosing of the vaporizable material. For example, the current subject matter includes vaporizer devices and methods of using such vaporizer devices for accurate and controlled dose delivery of an active ingredient in a vaporizable material (e.g., nicotine, cannabis, and any other active ingredient/drug) based on user specified, medical, dosing, or cessation needs. Dose configurations for a user may be controlled by another entity, for example, a health care provider, a friend or family member, and/or the like.

Information about the cartridge and/or a vaporizable material held in the cartridge may be particularly helpful in determining dose. For example information such as one or more of: the type of vaporizable material (e.g., nicotine, cannabis, etc.), the concentration of vaporizable material, the content of the vaporizable material, the amount of vaporizable material, the configuration of the cartridge (e.g., heater, electrical properties, etc.), the lot number of the cartridge, the date of manufacture of the cartridge, expiration date, the thermal properties of the vaporizable material, etc. may be used to accurately estimate dose. In some implementations of the current subject matter, dose and/or use information may be stored (written) on the cartridge (e.g., in a memory).

After completion of a vaporizer dose, it may be desirable to control the start of a new dose. In some aspects, the user may wish to limit or monitor the vaporizer dose for a given time period (e.g., doses per hour, day, week, month, etc.). In order to clearly distinguish vaporizer doses from one another, the vaporizer device and/or the user device may require a specific user input, wait time between doses, device setting or status, or other criteria before starting a new vaporizer dose.

Now turning to aspects of the current subject matter relating to vapor production by a vaporizer device, a model (herein also referred to as an analytical model, a machine learning model, and/or a statistical model) that predicts vapor production of a vaporizable material by a vaporizer device is provided. The determined vapor production is a function of various metrics relating to operation of the vaporizer device (e.g., sensor data and other parameters). As described herein, aspects of the current subject matter also relate to integration of the model into one or more systems to provide user control and visibility into the vapor production. For example, the model may be integrated into a connected device system to provide user control and visibility into the vapor production via one or more connected devices.

Consistent with implementations of the current subject matter, an amount of produced vapor may be determined. The amount of vapor produced may be used to allow for a user to be informed of an amount vaporized and/or consumed as well as optionally limit or otherwise control an amount vaporized and/or consumed.

For example, a dose control implementation consistent with implementations of the current subject matter provides for monitoring an amount of vapor produced against a desired or predefined dose or amount of vapor. When the amount of vapor produced equals or exceeds the desired or predefined dose, an indication may be sent to a user and/or the vaporizer device may cease operation for a period of time.

As another example, a fuel gauge implementation consistent with implementations of the current subject matter provides for monitoring an amount of vapor produced and providing to a user an indication of a remaining amount of vapor.

In accordance with some implementations of the current subject matter, a desired amount of vapor for production may be indicated or otherwise selected (e.g., user selected). The model consistent with implementations of the current subject matter may be used to determine when the desired amount of vapor is produced by the vaporizer device, which may be used to control operation of the vaporizer device (e.g., turn off the vaporizer device). The model consistent with implementations of the current subject matter may also be used to determine one or more settings associated with the vaporizer device to achieve the desired amount of vapor. The model consistent with implementations of the current subject matter may also be used to provide usage data related to use of the vaporizer device.

Implementations of the current subject matter provide for determining an amount of vapor consumed by or provided to a user and/or providing a predetermined or preselected amount of vapor through a vaporizer device. In accordance with implementations of the current subject matter, the amount of the vapor consumed by a user may be determined by taking into account various characteristics of a vaporizable material, the vaporizer device including the cartridge that delivers the vapor, and/or the user's behavior. Such determination allows for the user to limit or otherwise control an amount consumed, and also provides for consumption information (e.g., historical consumption information) to be generated and provided to the user.

While some implementations of the current subject matter may be described with respect to cannabis and cannabinoid-based vaporizable materials, for example cannabis oils, the disclosure is not limited to cannabis and cannabinoid-based vaporizable materials and may be applicable to other types of materials.

Historically, controlling and/or determining a consumption amount of cannabis has proven to be difficult. A pulmonary administration (e.g., through the lungs) of cannabis is typically achieved through combustion, which requires application of a high heat. Due to the high heat necessary to achieve combustion of cannabis, up to 30% (or in some instances an even larger percentage) of cannabinoids may be destroyed during combustion. Additionally, a further 10% up to 50% of the cannabinoids may dissipate through side stream smoke, which escapes from the vaporizer device and is not consumed by the user. With respect to cannabis plant material, various studies reveal high variability in detected plasma levels of cannabinoids of interest, such as tetrahydrocannabinol (THC) when standardized cannabis cigarettes are smoked under similar conditions. Large variability also exists in laboratory conditions using, for example, a smoking machine. In such conditions, concentrations of cannabinoids may vary by as much as 35%.

Cannabis concentrates, such as wax or oil, address some of the variability issues that occur with cannabis plant material. For example, unlike cannabis plant material, which has a highly variable distribution of cannabinoids, cannabis concentrates may achieve a uniform distribution of cannabinoids across a given sample. As such, the concentrations of cannabinoids may be known with a high degree of accuracy across an unlimited quantity of concentrate, provided that the batch for a given strain is well homogenized and tested for accuracy.

However, delivery of the concentrate to a user's lungs remains a source of variability in delivered amounts of cannabis concentrates. Different methods (e.g., dabbing, vaping) and different devices (e.g., cartridges from different manufacturers) may produce varying quantities of cannabis vapor and aerosol. Furthermore, depending on such factors including but not limited to a temperature of a heating element, an age of the cannabis concentrate, a type of wick and coil, a type of heating system, a flow rate of an inhalation draw, and/or the like, concentrations of cannabinoids delivered to the lungs may vary. This may be of particular relevance in the case of cannabis, as compared to other vaporizable materials, as cannabinoids work synergistically in the body to produce some of the medical and non-medical effects for which they are known. Thus, for example, variation in relative ratios of two or more cannabinoids and other psycho-active and bio-active compounds in cannabis may result in variation in medically and non-medically relevant effects.

Particle size of aerosols plays a role in determining whether and where a given particle is absorbed by the body. Cannabis concentrates produce a hetero-disperse aerosol, in which diameters of particles present in the aerosol may vary by orders of magnitude. Consequently, some particles interface with the body at various parts of the airways, while other particles are exhaled. The proportion of particles absorbed versus exhaled is an important determinant of the plasma concentration of cannabinoids (i.e., bioavailability). Particle size is influenced by a variety of factors, including the materials used to produce the aerosol (e.g., wick material and geometry, coil material and geometry), the heat applied to aerosolize the vaporizable material, the flow rate at which a user may draw, and/or the like.

Such variations in concentrations of cannabinoids delivered to the lungs and in particle size make estimation or determination of a quantity of cannabinoids delivered to the lungs challenging.

Consistent with implementations of the current subject matter, an amount of vapor consumed by a user is determined by taking into account factors including total particulate matter generated (which refers to the amount of vaporizable material removed from the wick by vaporization or aerosolization and suspended in the vapor), potency information (which refers to the chemical composition of the vaporizable material), temperature effects caused by a vaporizable material being heated, and/or particle size of aerosols generated (which refers to the particles that form the vapor). Information related to energy consumption, the vaporizable material from which the vapor is generated (e.g., inherent properties of the vaporizable material), usage history of a cartridge in which the vaporizable material is contained, ambient conditions, type of wick, type of heating technology, and/or the like may be relevant to the determination of the amount of vapor consumed.

As previously described herein, a user may apply dose control features, which limit the amount of energy supplied to the heater 166 to result in a controlled amount of vapor being produced from the vaporizable material. In accordance with some implementations of the current subject matter, available dose sizes signify or are representative of how much vapor is being produced for inhalation by a user. A strong, positive correlation exists between the amount of energy supplied and an amount of vaporizable material removed from the cartridge 150. The amount of the vaporizable material removed is referred to as total particulate matter (sometimes referred to as TPM). Without accounting for other factors, such as variations between cartridges, a model, such as a linear regression model of the total particulate matter fitted to the amount of energy supplied, may in some instances have a coefficient of determination (R²) of greater than 0.8. Therefore, with knowledge of the energy consumption recorded by the vaporizer device 100, the total particulate matter generated in a single puff or a series of puffs can be determined and thus provide an estimate or approximation of the total particulate matter.

Implementations of the current subject matter may provide for incorporating information or characteristics related to a user's puff (e.g., length of draw, length of time between puffs, number of puffs, and the like) and temperature information (e.g., a temperature preset by the user or set by the vaporizer device 100) to determine total particulate matter generated. Consistent with additional implementations of the current subject matter, information related to the vaporizable material may also be incorporated as described further herein. For example, ratios of various chemical constituents of the vaporizable material may provide for an estimation of a temperature-viscosity curve, which may be specific to the particular batch of the vaporizable material. Further, an estimate of the viscosity of the vaporizable material allows for determining a rate of wick re-saturation as well as determining a relationship between energy supplied to a heating element of the heater 166 and the generated total particulate matter. Additionally, consistent with implementations of the current subject matter, incorporating information or data related to ambient conditions (e.g., ambient temperature, pressure, and/or humidity) further improves accuracy of the determination of the total particulate matter generated. Further refinements in determining total particulate matter generation may be achieved by incorporating information related to an age of the vaporizable material, as this may affect physical and chemical properties of the vaporizable material (e.g., potentially including viscosity and average vapor pressure) in a predictable way that affects the rate of aerosolization. Additional and/or alternative refinements in determining total particulate matter generation may include accounting for wick and/or heating element properties, as wick type may affect re-saturation and desaturation rates and the type of heating technology employed also affects rate of total particulate matter generation.

Consistent with implementations of the current subject matter, an amount of vapor consumed by a user may be a percentage of the total particulate matter that is generated. As some vaporizable materials are complex mixtures of cannabinoids, terpenes, and other chemicals, an amount of a given cannabinoid delivered may be expressed as a percentage of the total particulate matter generated. For example, if it is estimated that the total particulate matter generated is 1 mg, it may be estimated that the total particulate matter for a concentrate that is 70% THC, assuming zero loss, is 70% THC; and the amount consumed may then be 700 μg. That is, with knowledge of the chemical composition of the contents of the cartridge 150, a basic estimate of the delivered amount of each chemical constituent of the concentrate may be made.

A temperature at which the vaporizable material is vaporized may also be used as a factor with the total particulate matter generated to determine the amount of vapor consumed. For example, ratios of cannabinoids, terpenes, and other chemicals in the vapor depend on the temperature at which the vaporizable material (e.g., the concentrate) was vaporized. Thus, the amount consumed estimation may be refined by accounting for the temperature preset or temperature of the heater at which the vaporizable material was vaporized. In some implementations, a default maximum temperature for a particular cartridge may be determined based on known temperature affects.

Dynamic (e.g., real-time or current) potency information may in some implementations of the current subject matter be determined and applied with the determined total particulate matter generated to provide a more accurate estimate of the amount of vapor consumed by the user. For example, with knowledge of the chemical composition of the vaporizable material, the date on which it was chemically analyzed, and/or the shelf stability of the various constituents of the vaporizable material, the current potency of the various constituents of the vaporizable material may be determined based on the chemical half-life of each constituent. That is, in some implementations factors related to chemical half-life of the various constituents are utilized to aid in determining a quantity of cannabinoids delivered to the user. In particular, the chemical half-life's effect on stability of a cannabis concentrate is taken into account. As cannabinoids degrade (and form new compounds, some of which are other cannabinoids) at a predictable rate, with knowledge of the chemical composition of the vaporizable material as measured on a specific date, the composition of the vaporizable material may be projected for a future date. The presence and potency of the various active compounds in the vaporizable material (e.g., a cannabis concentrate) may be used as a layer to interpret the potency of the vaporizable material as delivered to the user.

Dynamic potency consistent with implementations of the current subject matter may improve the user experience by giving the user additional insight into and control over consumption. Furthermore, dynamic potency, when layered onto aggregate data about consumption of a cartridge as well as user-generated feedback, may yield data-points for analysis, which may be used to explore the so-called “entourage effect” and may be used to identify combinations of cannabis constituents which may be candidate drug combinations.

Additionally, the dynamic potency estimation may be of interest to the user, and accordingly data indicative of the dynamic potency estimation may be provided to the user device 305 for display thereof to indicate the freshness of the cartridge and/or to make recommendations related to other cartridges for use by the user.

As previously described, the amount of vapor emitted by the cartridge is not the same as the actual amount delivered to the user. One of the factors influencing the actual amount delivered to the user is the aerosol particle size distribution. While larger particles may generally contain greater amounts of the vaporizable material, they are typically less efficient at penetrating deep into the lungs, where optimal drug delivery occurs. Thus, in order to estimate the bioavailability (which refers to the amount or proportion of the vaporizable material that enters the body) of a given aerosol, consistent with implementations of the current subject matter, particle size distribution is estimated and related to a rate of absorption in the user. This may be accomplished by correlating particle size distribution to factors that include but are not limited to chemical composition and the viscosity of the vaporizable material, ambient temperature, pressure and humidity, draw length, rate, and volume. Additionally, the total particulate matter may be correlated with the various factors.

Consistent with some implementations of the current subject matter, a user may indicate or select, via for example an application running on the user device 305 as described elsewhere herein, a desired amount of vapor to be consumed. Using the methods disclosed herein with respect to total particulate matter, dynamic potency, and/or bioavailability, the vaporizer device 100 may deliver the desired amount of vapor to the user.

As noted above, when a cartridge is removed from and re-inserted into the vaporizer body, the pre-selected or preconfigured parameters (e.g., those from a previous dose) may be automatically applied. Alternatively, the device may reset to baseline parameters, which may be user or system defined and/or customizable and which may be overridden.

Aspects of the current subject matter relating to predicting and/or determining an amount of vapor production of a vaporizable material by a vaporizer device are not limited to use with the particular and/or exact configurations and/or components of the vaporizer device 100, the vaporizer body 110, and the cartridge 150 described with reference to FIG. 1A-FIG. 3. Rather, aspects of the current subject matter may be employed with various other vaporizer devices, vaporizer bodies, and cartridges and/or with various modifications of the vaporizer device 100, the vaporizer body 110, and the cartridge 150 described herein. For example, consistent with implementations of the current subject matter, aspects of the current subject matter may be employed without the tag 164 of the cartridge 150. Moreover, various sensors and circuitry may not be required for the operations provided herein. For example, the ambient pressure sensor 138, the accelerometer 139, and/or the cartridge detection circuitry 148 may not be required in some implementations. Various other combinations of configurations and/or components of the vaporizer device 100, the vaporizer body 110, and the cartridge 150 may be employed consistent with implementations of the current subject matter.

Turning to the aspects of vapor production and predicting an amount of vapor production by a vaporizer device, a user may find it desirable to monitor and/or control consumption of the vaporizable material (e.g., contained in the cartridge 150). Such monitoring and/or controlling may beneficially allow the user to adjust an amount of vaporizable material available over a certain time period, a total time allowed or allotted for using the vaporizer device 100, a time period between vaporizer uses, other consumption settings to meet the needs of the user, and/or the like.

Aspects of the current subject matter are directed to predicting the amount of vapor produced by vaporization of a vaporizable material by a vaporizer device. Aspects of the current subject matter are also directed to producing a desired amount of vapor for consumption by the user. While some implementations of the current subject matter may be described with respect to cannabis and cannabinoid-based vaporizable materials, for example cannabis oils, the disclosure is not limited to cannabis and cannabinoid-based vaporizable materials and may be applicable to other types of materials.

The amount of vapor produced as used herein may refer to total particulate matter (TPM), which refers to the amount of vaporizable material removed (e.g., from the cartridge 150 from a wick or wicking element contained in the cartridge 150) by vaporization or aerosolization and suspended in the vapor for consumption by the user. Total particulate matter may be represented in units of mass, such as milligrams (mg).

According to aspects of the current subject matter, a model such as a machine learning model and/or a statistical model is trained and provided to predict an amount of vapor production, produced by the vaporizer device 100, as a function of various factors (or metrics) relating to operation of the vaporizer device 100 (e.g., sensor data and other parameters). For example, factors relating to the heater 166, the temperature at which the vaporizer device 100 is operating, and/or the user's use of the vaporizer device 100 (e.g., length of time during which the user is puffing or drawing from the vaporizer device 100, strength of the puff, etc.) may contribute to the amount of vapor production. A model consistent with implementations of the current subject matter is provided to take into account such features to predict the amount of vapor production of the vaporizable material. The model may then be used to provide vapor production information and/or usage control to the user so that the user is aware of and/or in control of consumption.

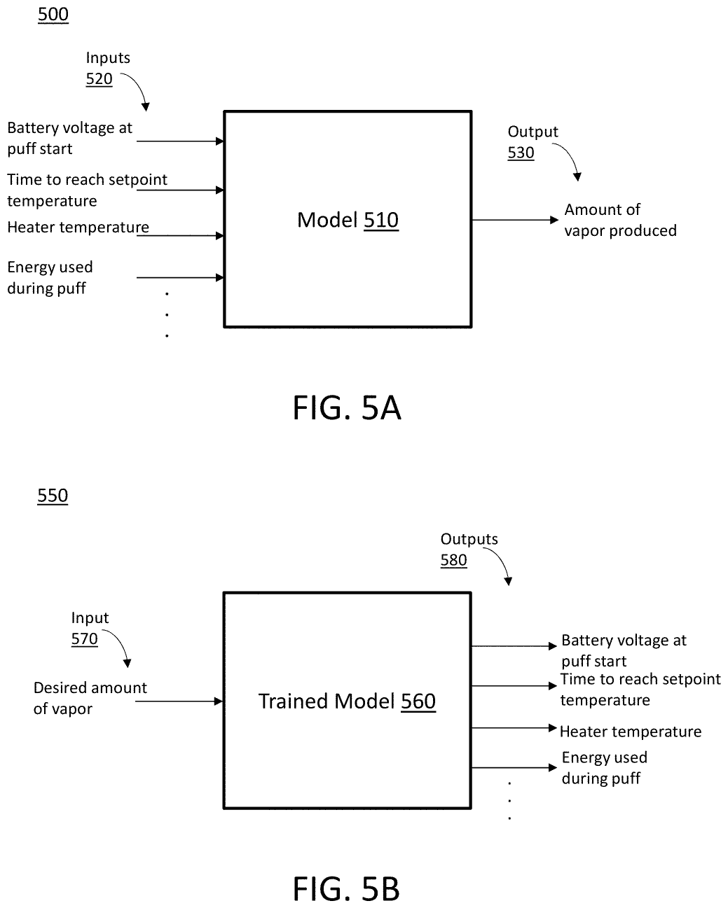

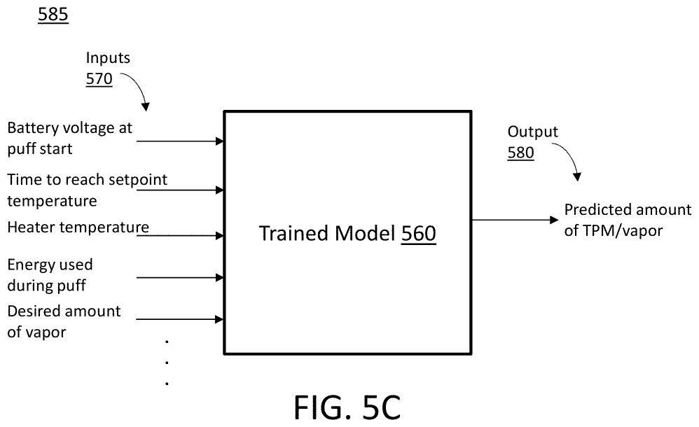

With reference to FIG. 5A, a diagram 500 depicts a training phase of a model 510 consistent with implementations of the current subject matter. As shown, inputted into the model 510 are a number of inputs 520 and an output 530. The inputs 520 and the output 530 may correspond to reference data, such as training data for the model 510 to learn the particular relationship between the inputs 520 and the output 530. In particular, the training data is utilized during the training phase so that the model 510 learns how to model the output 530 given the inputs 520, and how to model the inputs 520 given the output 530.

The inputs 520 may include, for example, energy used during puff, heater temperature, time to reach setpoint temperature, and battery voltage at a starting point of a puff. The inputs 520 may also include, for example, differential pressure integral (e.g., the integral of the pressure differential between the air path and ambient pressures), heater resistance, and other metrics relating to operation of the vaporizer device 100, although other types of inputs may be used as well. The output 530 is the amount of vapor produced (e.g., a value of total particulate matter). The total particulate matter is measured as a difference in mass of the cartridge 150 between puffs, with the reduction in mass of the cartridge 150 after each puff being equal to the amount of vapor produced. Using series of known values of the inputs 520 and the output 530, a variety of models may be trained such that the relationship between the inputs 520 and the output 530 is determined. Once trained, the model 510 is configured to receive the inputs 520 and generate a predicted value for the output 530. As more data becomes available (e.g., more inputs and a corresponding known output), the model 510 may continuously or periodically be trained based on new data points, resulting in a more refined predicted value for the output 530.

Consistent with implementations of the current subject matter, the model is trained on a training set, such as a collected dataset. The collected dataset may include data collected in a laboratory setting in a controlled environment, in which inputs such as energy, temperature, puff volume, and puff length are systematically varied in a factorial design.

According to an implementation of the current subject matter, to train the model, training data was collected over about 1,900 puffs on the vaporizer device 100. The puffs were conducted using a puffing machine in a factorial experiment which varied puff parameters such as temperature, puff length, and puff volume in a manner to simulate the breadth of user settings in the field. Each condition (e.g., combination of parameters) was replicated several times. Before and after each puff on the smoking machine, the mass of the cartridge 150 was measured, with the mass difference between puffs providing an amount of vaporizable material vaporized by each puff and representing the total particulate matter (TPM). As described, the machine learning model was trained to predict an amount of vapor production of a vaporizable material by the vaporizer device 100 during a user puff.

With reference to FIG. 5B, a diagram 550 depicts features of utilizing a trained model 560 consistent with implementations of the current subject matter. The trained model 560 (trained as described above with reference to FIG. 5A) may receive as an input 570 a desired amount of vapor to be produced. As the trained model 560 knows the relationship between metrics relating to operation of the vaporizer device 100 and the amount of vapor produced, the trained model 560 may take the amount of vapor to be produced (the input 570) and determine as outputs 580 the metrics relating to operation of the vaporizer device 100 (e.g., energy used during puff, heater temperature, time to reach setpoint temperature, battery voltage at a starting point of a puff, and other metrics relating to operation of the vaporizer device 100).

With reference to FIG. 5C, a diagram 585 depicts features of utilizing the trained model 560 consistent with additional implementations of the current subject matter. The trained model 560 (trained as described above with reference to FIG. 5A) may use as inputs 570 sampled data relating to operation of the vaporizer device 100 to generate a vapor production (e.g., TPM) prediction, the output 580. Thus, according to some aspects of the current subject matter, an output 580 of the trained model 560 may be a vapor production (e.g., TPM) prediction.

According to aspects of the current subject matter, a desired amount of vapor may also be one of the inputs 570. The vapor production prediction may be used in a dose control implementation and/or a fuel gauge implementation consistent with implementations of the current subject matter. For example, the predicted amount of vapor produces (the output 580 of the trained model 560) may be compared to the target or desired amount of vapor (e.g., one of the inputs 570). Once the target is reached, the heater 166 is, according to some implementations, turned off. In other implementations, the predicted amount of vapor is used to determine an amount of vapor remaining in the cartridge 150.

Consistent with implementations of the current subject matter, the model may be an adaptive boosted random forest regressor, which, on the collected dataset, may have high performance based on cross-validated root mean squared error (RMSE). A random forest regressor is an ensemble algorithm that averages predictions of many regression trees that are individually built on a random subset of the data and a random subset of the variables. The prediction performance of a random forest regressor is higher and more robust than that of individual regression trees. The act of averaging prevents overfitting of the training data, thereby improving the model's ability to generalize (e.g., make accurate predictions on unseen data). A random forest regressor is robust to noisy data such as outliers and multicollinearity (e.g., strong correlation between predictor variables). Adaptive boosting is a meta-algorithm that learns from its mistakes by giving more weight to regression trees that perform poorly. The result of incorporating adaptive boosting is an overall improved predictive accuracy.

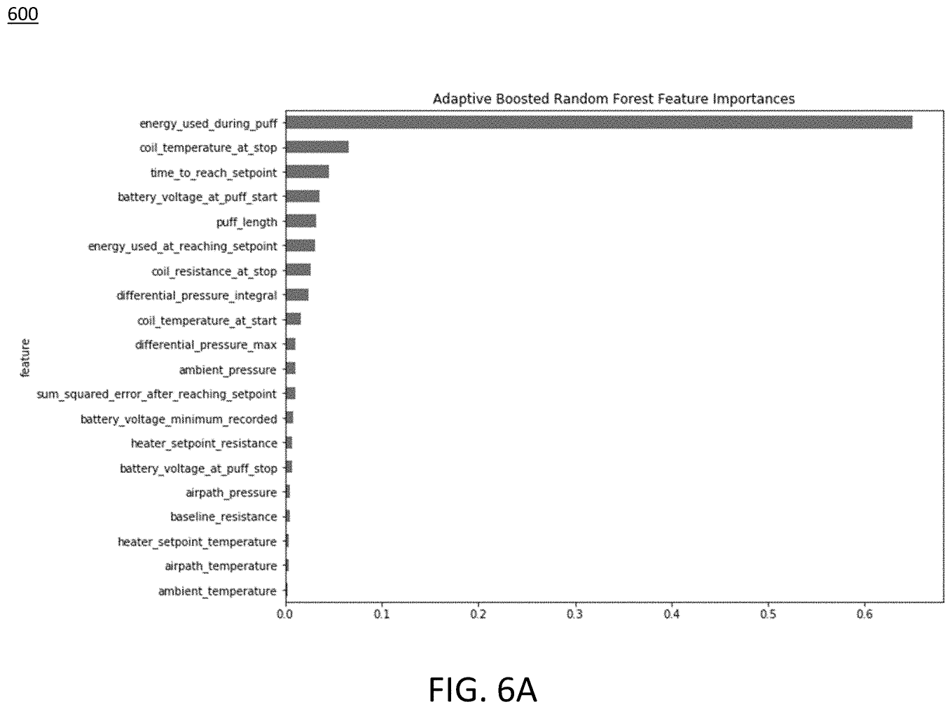

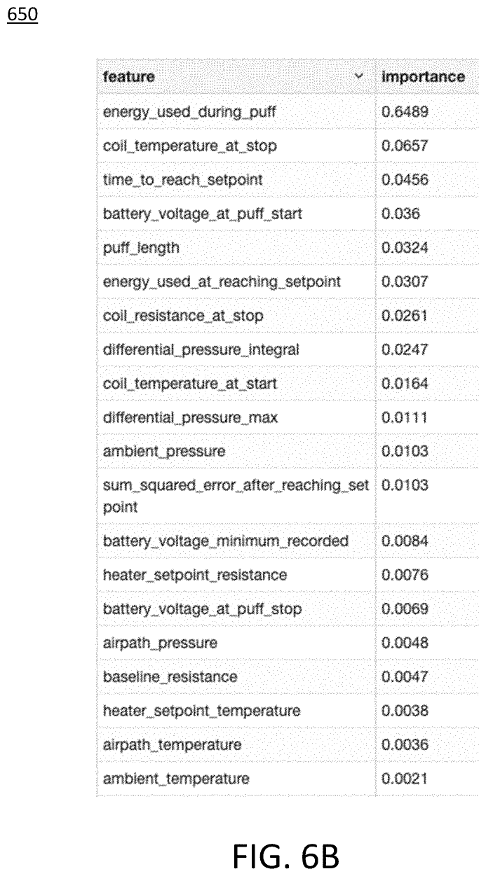

FIG. 6A is a chart 600 that illustrates feature importance of the inputs 520 (e.g., the operation metrics of the vaporizer device 100) as determined by a trained machine learning model in one example. FIG. 6B is a table 650 that provides importance values of the features for the example provided. Quantification of importance is, according to some aspects of the current subject matter, unitless and calculated from a mean decrease in impurity. Depending on the type of machine learning model, other features and importance qualifiers may be used, and aspects of the current subject matter are not limited to those shown in FIG. 6A and FIG. 6B.

The operation metrics of the vaporizer device 100 may include, but are not limited to: an amount of energy used during the user puff; a temperature of a heating element (e.g., the heater 166) of the vaporizer device 100 (e.g., a temperature at a start of the user puff, a temperature at an end of the user puff, and/or a setpoint temperature); a differential pressure value of the vaporizer device 100 (e.g., a different pressure integral and/or a maximum differential pressure); a resistance value of the heating element (e.g., the heater 166) of the vaporizer device 100 (e.g., a resistance value at an end of the user puff, a baseline resistance value, and/or a resistance value when the heater 166 is at a setpoint temperature); a battery voltage of the battery 124 of the vaporizer device (e.g., a battery voltage at a start and/or at an end of the user puff and/or a minimum recorded battery voltage); an amount of time to reach a setpoint temperature of the vaporizer device 100; an ambient pressure value; an amount of time of the user puff; an amount of energy to reach the setpoint temperature; an air path pressure in an air path of the vaporizer device 100; a sum squared error after reaching a setpoint temperature (e.g., the sum of the squared differences between setpoint temperature and heater temperature after a lower bound of the setpoint temperature has been crossed); an ambient temperature; and an air path temperature in the air path of the vaporizer device 100.

Consistent with additional implementations of the current subject matter, additional training data was collected over about 1,900 puffs on the vaporizer device 100, although other quantities of training data may be collected as well. The puffs were conducted using a puffing machine in a factorial experiment which varied puff parameters such as temperature, puff length, and puff volume in a manner to simulate the breadth of user settings in the field. Each condition (e.g., combination of parameters) was replicated several times. Before and after each puff on the smoking machine, the mass of the cartridge 150 was measured, with the mass difference between puffs providing an amount of vaporizable material vaporized by each puff and representing the total particulate matter (TPM).

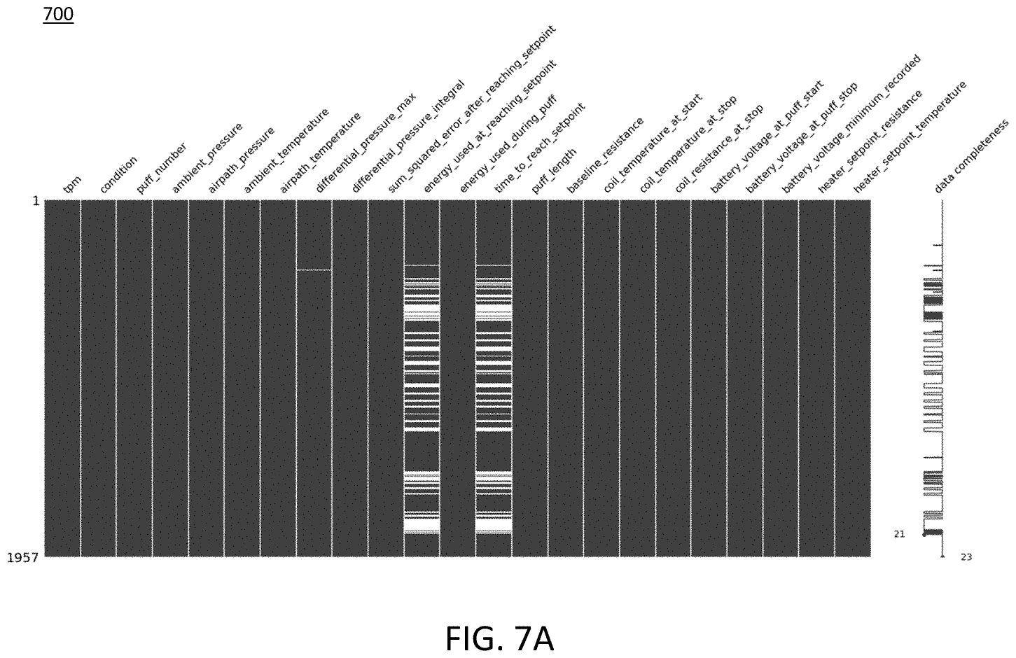

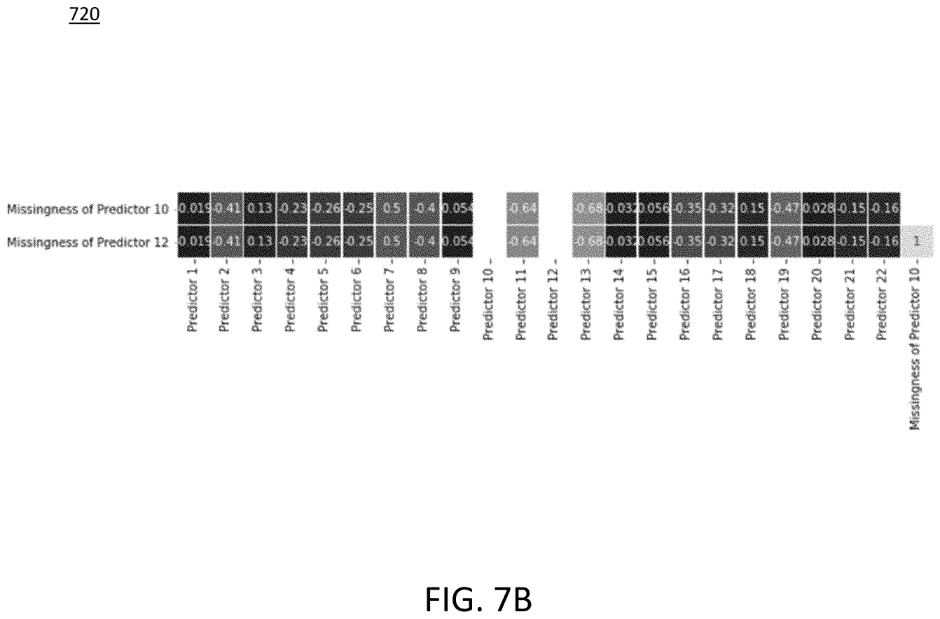

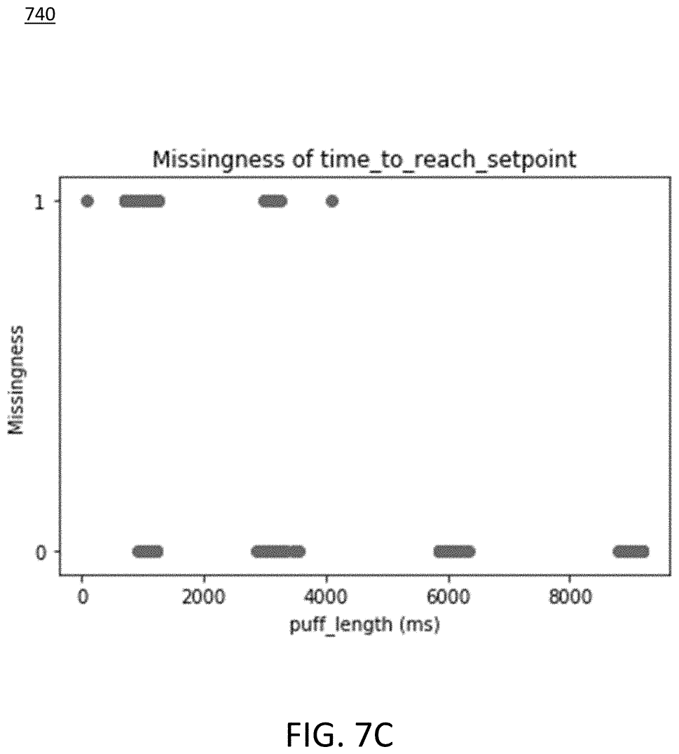

Exploratory data analysis was conducted to discover patterns and relationships between variables and to identify possible data anomalies for additional investigation. Missingness patterns in the data were assessed. A missingness matrix 700 from the Python library missingno, as shown in FIG. 7A, reveals that two variables (time to reach setpoint and energy used at reaching setpoint) seem to have values missing in chunks. A missingness heat map shown 720 in FIG. 7B indicates that the missingness between those two variables is perfectly correlated. The heat map 720 provides a correlation between the missingness of the two variables (e.g., whether or not each observation was missing) with each of the variables. Several moderately strong Pearson correlations suggest that these data could be missing at random (MAR), meaning that the probability of being missing can be described using the rest of the observed data. This hypothesis is supported by a scatter plot 740 between one of the missing variables and a moderately correlated variable, shown in FIG. 7C. The scatter plot 740 shows that time to reach setpoint is partly a function of puff length and is more commonly null at shorter puff lengths. This confirms that the time it takes for the heater 166 to reach the user-specified temperature setpoint would be right-censored by a short puff length (e.g., the heater 166 doesn't have time to reach the setpoint temperature for short puff lengths). A logistic regression was run to determine if the missingness could be accurately predicted from the rest of the data. A cross-validated regression model correctly predicted a missing value 95% of the time, confirming that the missingness was a function of the other variables in the dataset and not a function of the variable itself.

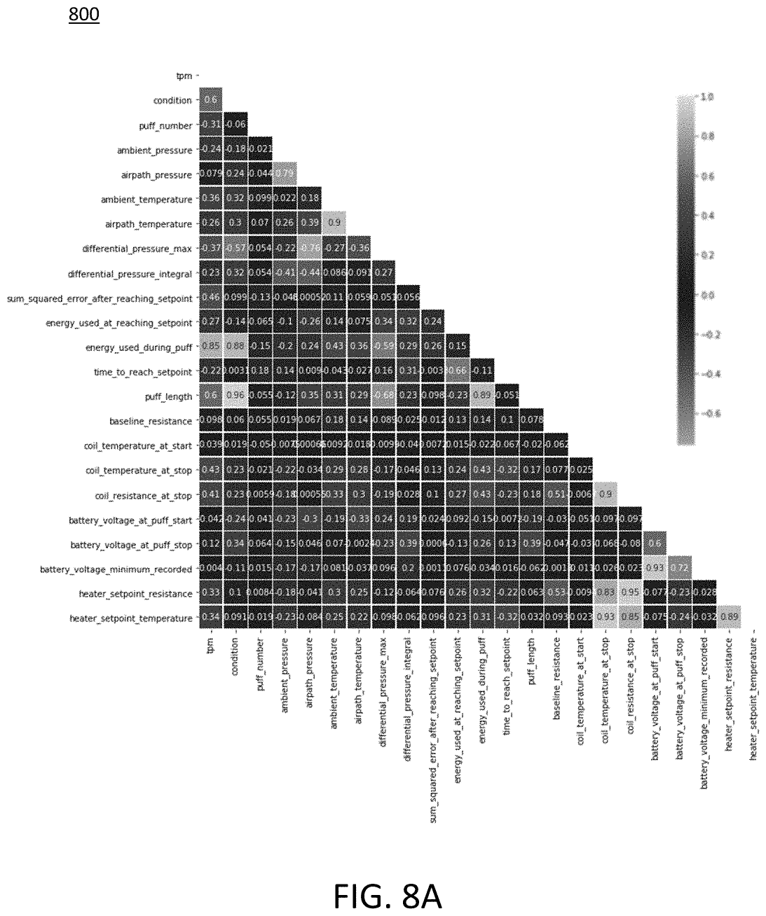

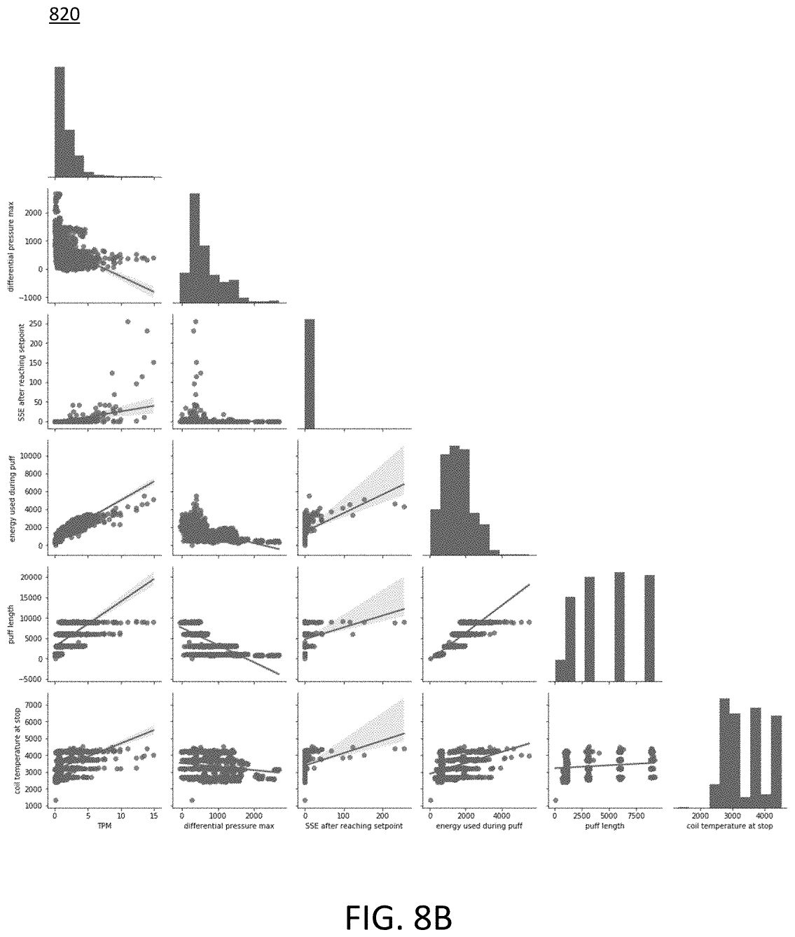

A correlation heat map and a pair plot were generated to understand how the variables relate to each other pairwise. The correlation heat map 800 shown in FIG. 8A is a seaborn heat map visualization of a Pearson correlation matrix, which shows the correlation between pairwise variables between −1 and 1. By looking at the heat map 800, the variables that are likely to influence the response variable in a regression are identified. It is also noted that there are quite a few high correlations between predictors in the matrix, which indicates that multicollinearity may be problematic. The correlation matrix was used to drop predictors that have very low correlation (e.g., absolute value less than 0.1) with the response variable.

A pair plot 820, shown in FIG. 8B, is another tool to visualize the distribution of variables and their pairwise relationships with other variables. A non-linear pattern in a scatter plot, for example, may indicate that a polynomial term should be added to the regression.

A multiple linear regression with all predictors yields a very high condition number, thereby confirming that multicollinearity in the data is high. To identify the strongest contributors to multicollinearity, the variance inflation factor (VIF) for each predictor was calculated. The VIF quantifies how much the variance of each variable's regression coefficient would be inflated by the presence of correlation with other predictor variables. The variance inflation factor function from statsmodels was used to calculate the VIF for each variable as such:

Code Snippet 1: Calculating variance inflation factor

def calculate_vif(df):

vif = pd.DataFrame( )
vif[ [symbol] VIF [symbol] ] = [variance_inflation_factor(df.values, i)

for i in range(df.shape[1])]

vif[ [symbol] features [symbol] ] = df.columns
vif = vif.round(1)
print(vif.sort_values( [symbol] VIF [symbol] , ascending=False [symbol] ) [symbol]
indicates data missing or illegible when filed

This code returns a VIF value for each variable. A VIF greater than five generally indicates strong collinearity with other variables and may warrant further investigation, while a VIF exceeding 10 may be a sign of serious multicollinearity that may require correction. Multicollinearity can be problematic because it leads to unstable estimates of regression coefficients and inflates standard errors. However, it does not bias predictions. The data set yielded very high multicollinearity for several variables, so variable selection was conducted for the linear regression case to reduce multicollinearity, improve confidence intervals around the regression coefficients, and to reduce the complexity of the model to meet engineering requirements.

Multicollinearity for the linear regression case was reduced by eliminating variables one at a time using VIF and domain knowledge. There are a handful of variables in the raw dataset that essentially capture the same information, for example, heater setpoint resistance and heater setpoint temperature are very similar because the heater temperature is calculated in part from the heater resistance. This means that the two variables significantly inflate the variance of each other, and removing one of them would greatly reduce multicollinearity without biasing the regression. Therefore the approach was to identify pairs or groups of such similar variables with high VIF, then remove the variable with a lower correlation with the response variable. Doing so iteratively drastically reduced the VIFs in the regression variables.

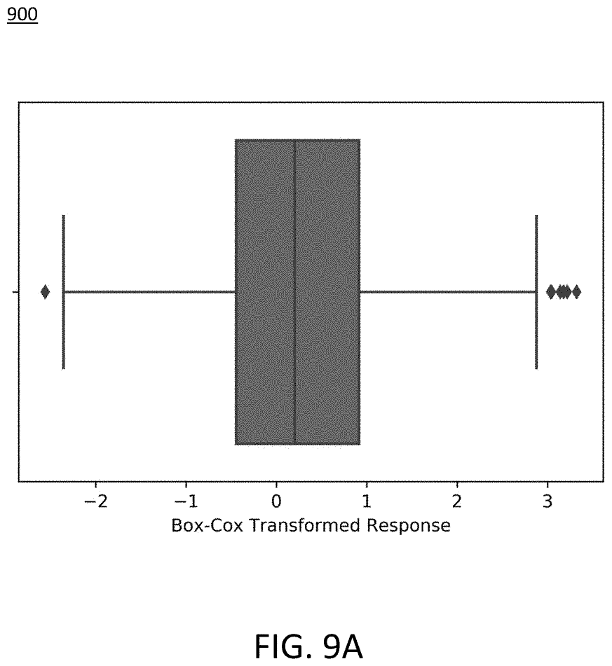

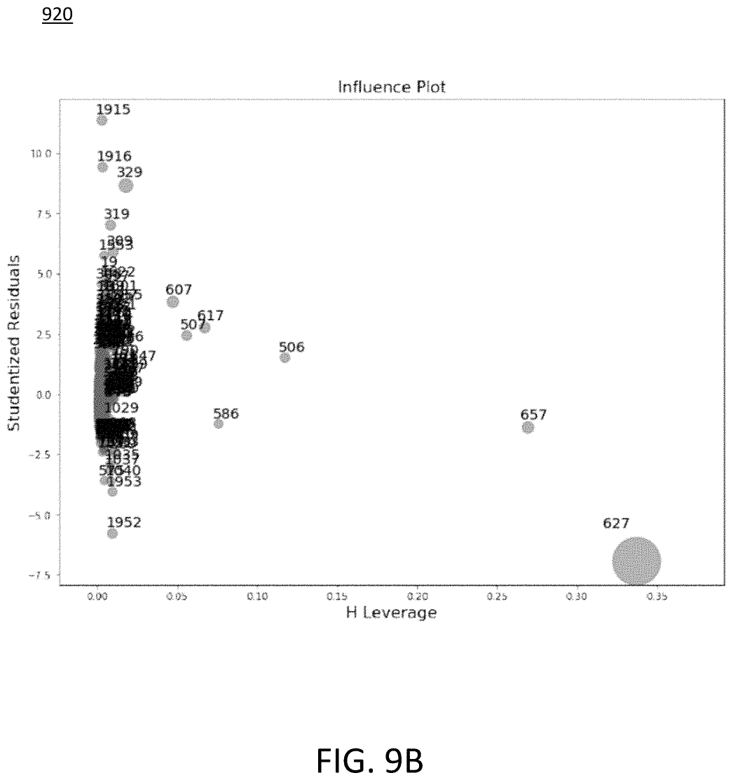

The regression analysis consistent with implementations of the current subject matter includes influential point detection and analysis. To identify univariate outliers, a box-and-whisker plot was used to quickly visualize extreme values of the Box-Cox transformed response variable. The Box-Cox transformation was used to correct for heteroscedasticity in the observed values. The Box-Cox transforms the distribution to reduce skewness and make it approximately normal. This makes the box plot approximately symmetrical and makes it easier to identify potential outliers. A box plot 900 in FIG. 9A illustrates that there are a few observations outside of the whiskers, demonstrating 1.5× the width of the interquartile range (IQR). These points may warrant further investigation to determine if they are problematic outliers that need to be addressed or simply valid, extreme values.

The influence of each data point on the regression was evaluated to assess the impact of extreme or unusual observations in a multivariate space. Influence is the combination of leverage (e.g., distance from the mean predictor values) and standardized residual values (e.g., distance from the mean response value) and is measured by Cook's distance. Cook's distance captures how much the regression model changes when the observation is removed from the regression. A Cook's distance greater than three times the mean is considered influential and may warrant additional investigation to determine its validity. Cook's distance may be represented as follows:

$${D}_{i} = \frac{{\sum }_{j = 1}^{n} {( {\hat{y}}_{j} - {\hat{y}}_{j ( i )} )}^{2}}{( p + 1 ) {\hat{σ}}^{2}}$$

Cook's distance can be visualized using statsmodels's influence plot, on which the Cook's distance is reflected in the size of the point, and the position of the point communicates the studentized residual and leverage. Thus, the plot quickly summarizes the Cook's distance, residual, and leverage of each point and thereby helps identify which points are potentially problematic and why. An example influence plot 920, generated for a particular model, is shown in FIG. 9B.

According to aspects of the current subject matter, and based on the influential points identified in the influence plots, such as the box plot 900 and the influence plot 920, observations from the analysis were not removed. None of the influential points indicated errors in the data itself (such as by recording or measurement errors); more data may be completed to meet the accuracy target, and additional data may reduce the peculiarity of the high influence points by improving the model fit; and the experiment was designed to cover the entire range of possible device settings (e.g., settings of the vaporizer device 100) and usage behavior of the vaporizer device 100; therefore, the model should be able to predict even unusual combinations of predictors. Consistent with implementations of the current subject matter, influential data points may be used to identify experiment conditions (e.g., combinations of predictors) for additional data collection purposes.

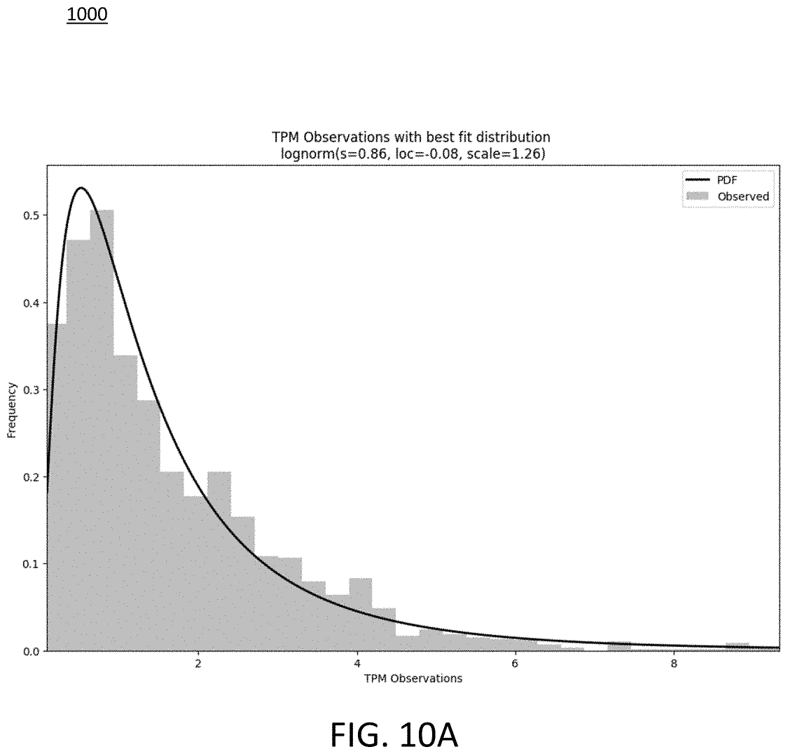

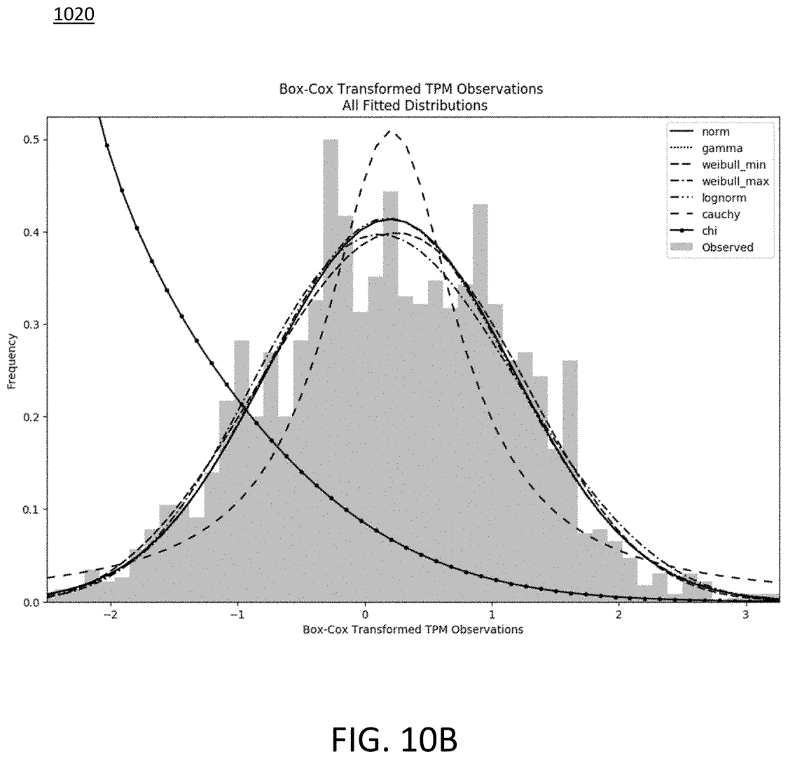

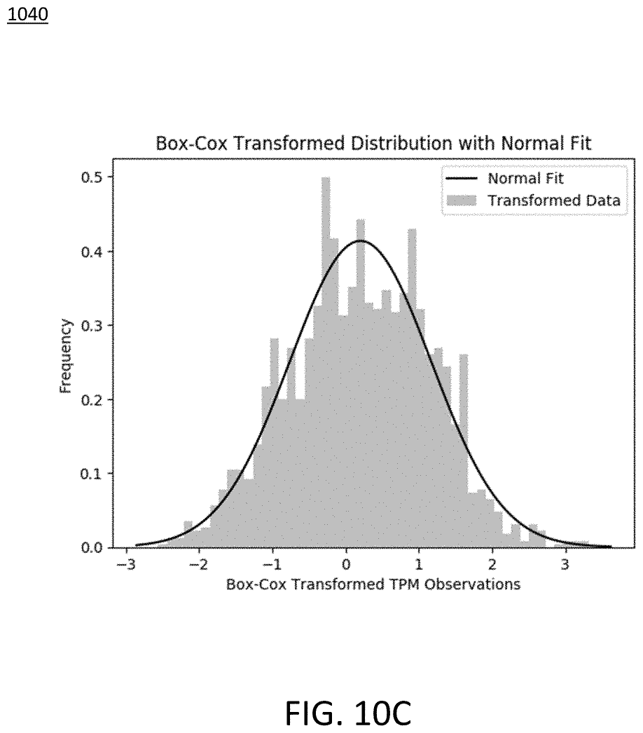

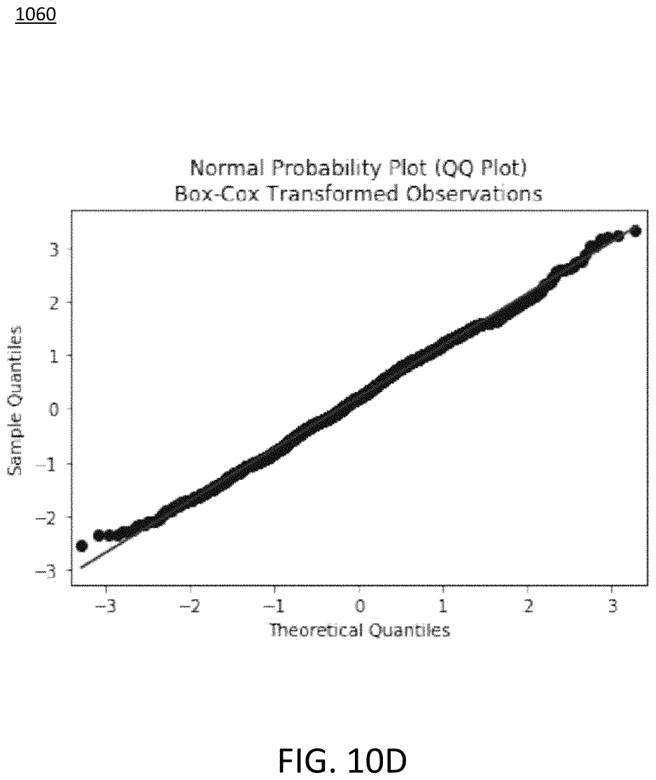

Histogram 1000, shown in FIG. 10A, illustrates the distribution of the response variable as a histogram of the observations, which reveals that the data has significant right skewness and is always positive. The skewness indicates that the variance is not constant—it is lower at smaller values of TPM and higher at the larger values—and therefore a regression of the data is likely to display heteroscedasticity in its residuals. This suggests that a transformation of the response variable would improve the model fit.

A Box-Cox transformation was applied to the response to correct the skewness. A Box-Cox is a family of transformations that transforms non-normal data into a normal distribution to meet the assumptions for a variety of statistical tests and techniques. The transformed data was fitted to a variety of known distributions, as shown in 1020 of FIG. 10B. As shown, the data appears approximately normal, as shown in 1040 of FIG. 10C. A quantile-quantile (QQ) plot 1060 of a normal distribution, shown in FIG. 10D, confirms that the transformed data is approximately normal. A Shapiro-Wilk normality test, however, rejects the null hypothesis of normality, suggesting that the distribution is not normal. A normal distribution of the response variable is not a requirement for linear regression, and the approximate normal distribution of the transformed data will help meet the linear regression assumptions of linearity, independence, homoscedastic residuals, and normally-distributed residuals.

According to aspects of the current subject matter, a prototype analytic framework is provided for performing a variety of preprocessing steps and training a number of candidate regression models on the training data. The framework was built in a series of Apache Zeppelin notebooks using Python and the Python-based machine learning library scikit-learn.

The analytic framework leverages scikit-learn's Pipeline class, which sequentially applies a series of transformations to a dataset prior to the final estimator. Each candidate model is fitted in the final step of a pipeline and is preceded by the following preprocessing steps: standardization, power transformer, polynomial features, and feature selection. Standardization is necessary for regularization regression models, such as lasso, ridge, and elastic net. These models apply a regularization penalty to variables based on the magnitude of their coefficients. If variables are on different scales, the penalty will unevenly penalize variables according to their scale (with smaller-scaled variables being penalized more). For example, a temperature variable measured in degrees Celsius will be more heavily penalized than an energy variable measured in milli-joules due to its relatively larger coefficient. Standardization before regression puts all variables on the same scale and therefore equal footing.

Scikit-learn's PowerTransformer class may be used to automatically transform each predictor variable using a Box-Cox or Yeo-Johnson transformation to reduce non-normality in the predictors. This in turn helps reduce heteroscedasticity, or non-constant variance throughout the variable's entire range, in the residuals.

Scikit-learn's PolynomialFeatures may be used to programmatically include quadratic terms and interaction variables for linear regression. These extra terms may help explain non-linear relationships between variables.

In addition to the manual process of eliminating variables using VIF and domain knowledge, programmatic variable selection techniques may be applied to reduce the dimensionality of the data after including quadratic and interaction terms for linear regression. SequentialFeatureSelector from the mlxtend library may be used to perform forward stepwise selection, and RFECV from scikit-learn may be used to perform recursive feature elimination (or backward stepwise selection).

Consistent with implementations of the current subject matter, variable selection for the regularization methods, including lasso, ridge, and elastic net, may be different. These models intrinsically perform variable selection, so a separate variable selection step to the pipelines for these models may not need to be applied. Instead, scikit-learn's cross-validation variants of these classes are allowed to iterate over a range of regularization parameters (referred to as alpha in scikit-learn), then the optimal value is used to penalize the regression coefficients using the relevant distance norm.

According to aspects of the current subject matter, the prototype analytic framework loops through a Python dictionary of statistical regression models, adding each model to a pipeline with the aforementioned preprocessing steps. A sample of this dictionary is shown in code snippet 2 below. Scikit-learn's cross-validation classes LassoCV, RidgeCV, and ElasticNetCV may be used to optimize the regularization parameter. For each model in the dictionary of candidate models, the model is fitted both without transforming the response variable and with a Box-Cox transformation to the response variable. See Table 1 for a partial list of combinations of candidate regression models and transformations that were trained.

Code Snippet 2: Candidate regression models

regression_models = [
{ [symbol] name [symbol] : [symbol] linear reg [symbol] , [symbol] model [symbol] : LinearRegression(fit_intercept=True,

normalize=False)},

{ [symbol] name [symbol] : [symbol] lasso cv, normalized [symbol] , [symbol] model [symbol] :
LassoCV(fit_intercept=True, normalize=True, cv=10)},
{ [symbol] name [symbol] : [symbol] ridge cv, normalized [symbol] , [symbol] model [symbol] : RidgeCV(alphas=10**np.

linspace(10,−2,50)*0.5, fit_intercept=True, normalize=True, cv=10)
},

{ [symbol] name [symbol] : [symbol] elastic-net cv, normalized [symbol] , [symbol] model [symbol] : ElasticNetCV(l1_ratio

=[.01, .05, .1, .3, .5, .7, .9, .95, .99, 1], fit_intercept=True,
normalize=True, cv=10)},

{ [symbol] name [symbol] : [symbol] pca pipeline [symbol] , [symbol] model [symbol] : Pipeline(steps=[( [symbol] pca [symbol] , PCA( )), (

[symbol]

linear [symbol] , LinearRegression(fit_intercept=True, normalize=True))])}, [symbol]
indicates data missing or illegible when filed

TABLE 1
Candidate pipelines

| y- | X- | Polynomial | Feature | Regression |
|---|---|---|---|---|
| transfonn | transform | expansion | selector | model |
| None | StandardScaler | TRUE | None | LinearRegression |
| None | StandardScaler | TRUE | Forward | LinearRegression |
| None | StandardScaler | TRUE | Backward | LinearRegression |
| None | StandardScaler | FALSE | None | LinearRegression |
| None | StandardScaler | FALSE | Forward | LinearRegression |
| None | StandardScaler | FALSE | Backward | LinearRegression |
| None | Yeo-Johnson | TRUE | None | LinearRegression |
| None | Yeo-Johnson | TRUE | Forward | LinearRegression |
| None | Yeo-Johnson | TRUE | Backward | LinearRegression |
| None | Yeo-Johnson | FALSE | None | LinearRegression |
| None | Yeo-Johnson | FALSE | Forward | LinearRegression |
| None | Yeo-Johnson | FALSE | Backward | LinearRegression |
| Box-Cox | StandardScaler | TRUE | None | LinearRegression |
| Box-Cox | StandardScaler | TRUE | Forward | LinearRegression |
| Box-Cox | StandardScaler | TRUE | Backward | LinearRegression |
| Box-Cox | StandardScaler | FALSE | None | LinearRegression |
| Box-Cox | StandardScaler | FALSE | Forward | LinearRegression |
| Box-Cox | StandardScaler | FALSE | Backward | LinearRegression |
| Box-Cox | Yeo-Johnson | TRUE | None | LinearRegression |
| Box-Cox | Yeo-Johnson | TRUE | Forward | LinearRegression |
| Box-Cox | Yeo-Johnson | TRUE | Backward | LinearRegression |
| Box-Cox | Yeo-Johnson | FALSE | None | LinearRegression |
| Box-Cox | Yeo-Johnson | FALSE | Forward | LinearRegression |
| Box-Cox | Yeo-Johnson | FALSE | Backward | LinearRegression |
| None | StandardScaler | FALSE | None | LassoCV |
| None | Yeo-Johnson | FALSE | None | LassoCV |
| Box-Cox | StandardScaler | FALSE | None | LassoCV |
| Box-Cox | Yeo-Johnson | FALSE | None | LassoCV |
| None | StandardScaler | FALSE | None | RidgeCV |
| None | Yeo-Johnson | FALSE | None | RidgeCV |
| Box-Cox | StandardScaler | FALSE | None | RidgeCV |
| Box-Cox | Yeo-Johnson | FALSE | None | RidgeCV |
| None | StandardScaler | FALSE | None | ElasticNetCV |
| None | Yeo-Johnson | FALSE | None | ElasticNetCV |
| Box-Cox | StandardScaler | FALSE | None | ElasticNetCV |
| Box-Cox | Yeo-Johnson | FALSE | None | ElasticNetCV |

According to aspects of the current subject matter, the cross-validation technique is used to measure prediction errors on each pipeline to ensure that the final regression model will perform well on unseen data. Cross-validation minimizes overfitting and minimizes the bias-variance trade-off (captured in the equation for mean squared error) by repeatedly fitting the model on a subset of the data, then averaging the prediction error to get an unbiased estimate of the model's ability to generalize. A method of cross-validation, such as k-fold cross-validation, which divides the data into k subsets or “folds” and cycles through them, may be used. For each of the k folds: the regressor is trained on the other k−1 folds, and predictions are made and error calculated on the current validation fold.

The prediction error is averaged across the k folds to arrive at an overall, cross-validated error measurement for the model. For example, the training data is divided into 10 folds, and for each fold a linear regression is fitted, resulting in 10 fitted linear regressions, 10 error calculations, and one final cross-validated error calculation. At the end of cross-validation, the model may be retrained on all of the training data for downstream use.

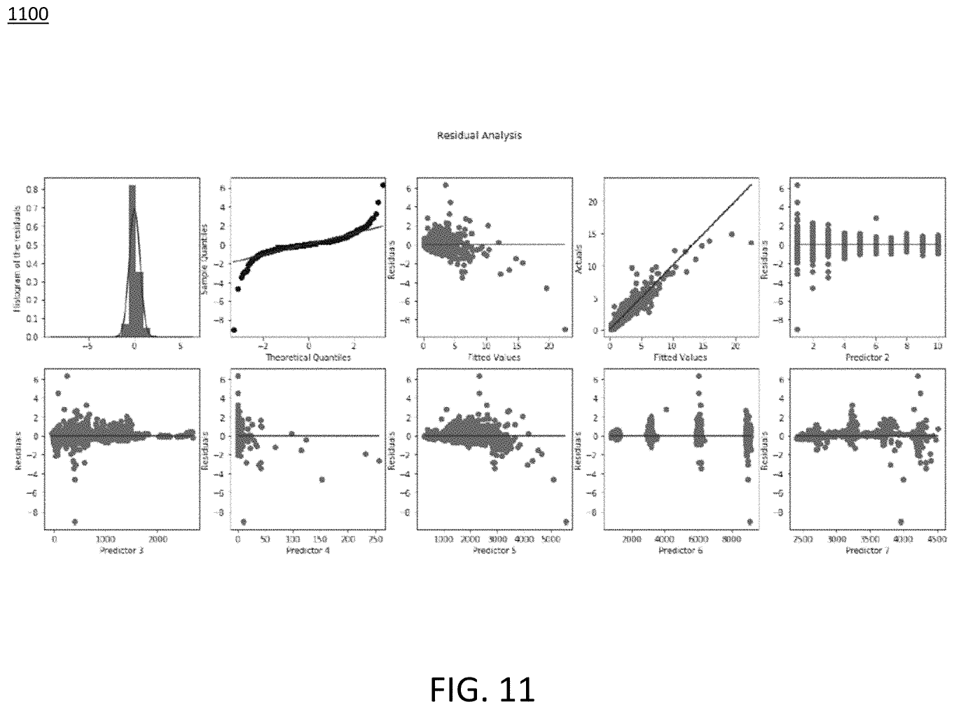

After fitting each of the candidate models, the residuals of each candidate model are, according to aspects of the current subject matter, analyzed to assess the model's fit with respect to the linear regression assumptions: linearity, independence, homoscedasticity, and normally distributed residuals. A series of residual plots using the predicted and observed values may be utilized to visually characterize the quality of the model fits. For each fitted model, the following plots may be generated. A histogram plot of the residuals may be generated (fitted to a normal curve for comparison) to assess the normality of the residuals. A linear regression assumes the residuals follow a normal distribution with zero mean. A quantile-quantile (QQ) plot may be generated; this plot provides another way to evaluate the normality of the residuals. Quantiles from the residuals are plotted against the quantiles from a theoretical normal curve. Normally-distributed residuals should on average adhere to the 45 degree line, so any departure indicates a violation of this assumption. A residuals versus fitted values plot may be generated. This multipurpose plot is used to evaluate the assumptions of linearity, independence, and homoscedasticity. Residuals from a good fit will be randomly distributed around the zero line with constant variance for all fitted values. An observed values versus fitted values plot may be generated. This is similar to the residuals vs. fitted plot but another way of visually assessing the assumptions of linearity, independence, and homoscedasticity. The residuals should be randomly distributed around the 45 degree line. Curves that deviate from the 45 degree line may help identify polynomial or interaction terms that are missing from the model. A residuals versus predictors plots may be generated. The residuals are plotted as a function of each predictor, and should be randomly distributed around the zero line with constant variance. If the plot does not uphold the assumptions of linearity and homoscedasticity, that implies that the predictor should be transformed and/or a polynomial or interaction term is missing. Residual plots 1100, consistent with implementations of the current subject matter, are shown in FIG. 11. According to aspects of the current subject matter, a Shapiro-Wilk normality test may be performed on each set of residuals to numerically determine if the residuals are normally distributed per the normality assumption. Violations of the normality assumption do not bias predictions.

The following types of regression models may be trained on the training data: linear regression, lasso regression, ridge regression, and elastic net regression. Consistent with implementations of the current subject matter, other types of regression models may also be trained for predicting vapor production. In some implementations, data transformations and feature selection methods may be applied to the trained models.

According to aspects of the current subject matter, a mean absolute percentage error (MAPE) may be used as an error metric to compare the candidate models. MAPE is an intuitive, percentage-based error metric for communicating results. The common pitfall of MAPE, that it cannot be calculated when an observed value is equal to zero, does not apply in this case because the TPM vaporized by a puff is always strictly positive. A synthesized sample of the model results are summarized in Table 2. Cross-validated MAPE is used to compare model performance, where lower MAPE indicates a better fit to the data.

TABLE 2
Sample cross-validated results

| | | | Polynomial | | | | Model |
|---|---|---|---|---|---|---|---|
| | | | & interaction | Feature | | | size |
| Index | y-transform | x-transform | terms | selector | Estimator | MAPE | (bytes) |
| 1 | b [symbol] | s [symbol] | True | Forward | LinearRegression | 20.2% | [symbol] |
| 2 | b [symbol] | s [symbol] | True | Backward | LinearRegression | 19.3% | [symbol] |
| 3 | b [symbol] | s [symbol] | True | None | LinearRegression | 17. [symbol] % | [symbol] |
| 4 | b [symbol] | y [symbol] | True | Forward | LinearRegression | 17.1% | [symbol] |
| 5 | b [symbol] | y [symbol] | True | Backward | LinearRegression | 15.0% | [symbol] |
| 6 | b [symbol] | y [symbol] | True | None | LinearRegression | 12.2% | [symbol] |
| 7 | b [symbol] | s [symbol] | None | None | [symbol] CV | 20.8% | [symbol] |
| 8 | b [symbol] | s [symbol] | None | None | RidgeCV | 24.7% | [symbol] |
| 9 | b [symbol] | s [symbol] | None | None | ElasticNetCV | 21. [symbol] % | [symbol] |

[symbol]

indicates data missing or illegible when filed

According to some implementations of the current subject matter, a consumer-facing application of the regression model (e.g., real-time use of the application in the vaporizer device 100) may require use of a statistical model to quantify TPM based on input data, as described herein. According to some aspects, a linear regression model with Yeo-Johnson transformed predictor variables and quadratic and interaction terms may be used for the prediction of vapor production implementations.

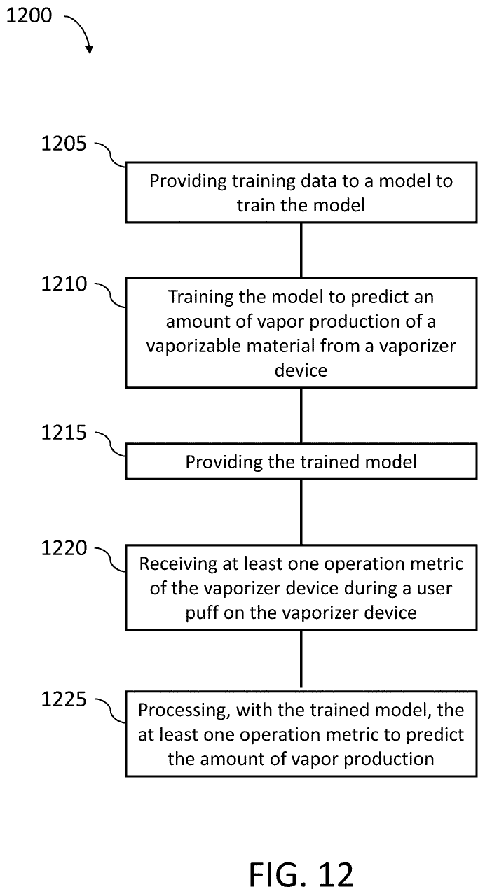

With reference to FIG. 12, a process chart 1200 illustrates features of a method, which may optionally include some or all of the following.

At 1205, training data is provided to train a model (e.g., a machine learning model and/or a statistical model) to predict, during a user puff on the vaporizer device 100, an amount of vapor production of a vaporizable material from the vaporizer device 100. The training data may include a plurality of operation metrics known to correspond to the amount of vapor production by the vaporizer device 100. For example, the training data may include inputs such as energy, temperature, puff pressure, and puff length. Other inputs may also be included as described herein. The training data also includes the corresponding output of the amount of vapor produced as represented as, for example, a value of total particulate matter.

At 1210, the model is trained to predict, during a user puff on the vaporizer device 100, the amount of vapor production of a vaporizable material from the vaporizer device 100. According to aspects of the current subject matter, the training of the model includes processing, with the model, training data as described with reference to FIG. 5A. For example, the inputs 520 and the output 530 are the training data for the model 510 to learn the particular relationship between the inputs 520 and the output 530. In particular, the training data is utilized during the training phase so that the model 510 learns how the output 530 is affected given the inputs 520. As described, the training data are systematically varied in the training of the model in an iterative process. The training data may include a plurality of values from a plurality of user puffs on the vaporizer device 100.

At 1215, the trained model is provided such that the trained model may be used to predict the amount of vapor production. The trained model may be provided for implementation at the vaporizer device 100, the user device 305, and/or the remote server 307.

At 1220, at least one operation metric of the vaporizer device 100 is received at the trained model from the vaporizer device 100. For example, operation metrics may include parameters or variables defining operation of one or more components of the vaporizer device 100 and may be related to the heater 166 and/or the battery 124. The operation metrics may be related to temperature values, energy values (e.g., energy supplied to the heater 166), and/or resistance values (e.g., resistance of the coil of the heater 166). The operation metrics may include one or more of the following: an amount of energy used during the user puff; a temperature of a heating element of the vaporizer device; a differential pressure value of the vaporizer device; a resistance value of the heating element of the vaporizer device; a battery voltage of a battery of the vaporizer device; an amount of time to reach a setpoint temperature of the vaporizer device; an ambient pressure value; an amount of time of the user puff; an amount of energy to reach the setpoint temperature; an air path pressure in an air path of the vaporizer device; an ambient temperature; and an air path temperature in the air path of the vaporizer device.

At 1225, the at least one operation metric of the vaporizer device 100 (collected from the vaporizer device 100 during the user puff on the vaporizer device 100) is processed with the trained model. The number of inputs for the trained model may be fewer than those used as input for the training of the model. By processing the at least one operation metric, the trained model is able to predict the amount of vapor production of the vaporizable material from the vaporizer device 100 during the user puff. According to aspects of the current subject matter, by using one or more operation metrics having a larger coefficient or importance value (e.g., see FIG. 6A and FIG. 6B), the trained model is able to make a more accurate prediction than with one or more operation metrics having a smaller coefficient or importance value. For example, it can be seen from the importance value data, that the energy used during a puff is an important metric in determining vapor production.

Consistent with implementations of the current subject matter, the model may be a machine learning model or a statistical model. The model may be a regression model. The model may be an adaptive boosted random forest regression model, an extra trees regression model, a gradient boosting regression model, a random forest model, a linear regression model, a ridge regression model, an elastic net regression model, a lasso regression model, a k-nearest neighbors regression model, a support vector regression, or other regression methods, and/or models. Aspects of the current subject matter are not limited to these particular types of models, and other types of models may also be used.

Consistent with implementations of the current subject matter, the trained model may be integrated into a system whereby receiving an indication of a selected amount of vapor, the model may then determine one or more operational settings of the vaporizer device 100 to achieve the selected amount of vapor. For example and with reference to FIG. 3, the trained model consistent with implementations of the current subject matter may be implemented at the remote server 307, at the user device 305, and/or at the vaporizer device 100. Through the application software (“app,” e.g., a mobile app and/or web app) running on the user device 305 and/or through the vaporizer device 100, the user may configure and/or control operational aspects of the vaporizer device 100 and receive information relating to operation of the vaporizer device 100. For example, the app may provide the user with capabilities to input or set a desired dose, the selection of which is, consistent with some implementations of the current subject matter, communicated to the remote server 307. In some implementations, the desired dose may be a dose size with a fixed amount of total particulate matter, and the user may be able to select the quantity (e.g., one, two, three, four, or more) of doses.

By running the trained model with the desired dose, a determination may be made as to one or more operational settings of the vaporizer device 100 to achieve the desired dose. The remote server 307 may send the one or more operational settings to the user device 305, which may then send the one or more operational settings to the vaporizer device 100. In some implementations, the remote server 307 may communicate directly with the vaporizer device 100. In some implementations, the remote server 307 is not needed, and the trained model may be implemented on the user device 305 and/or the vaporizer device 100.

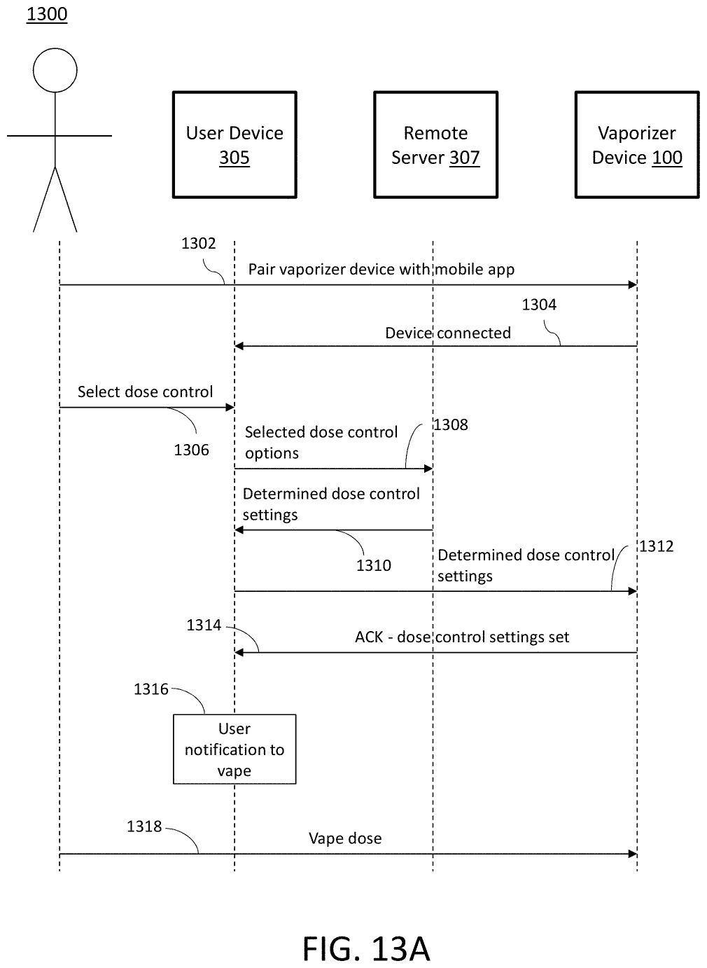

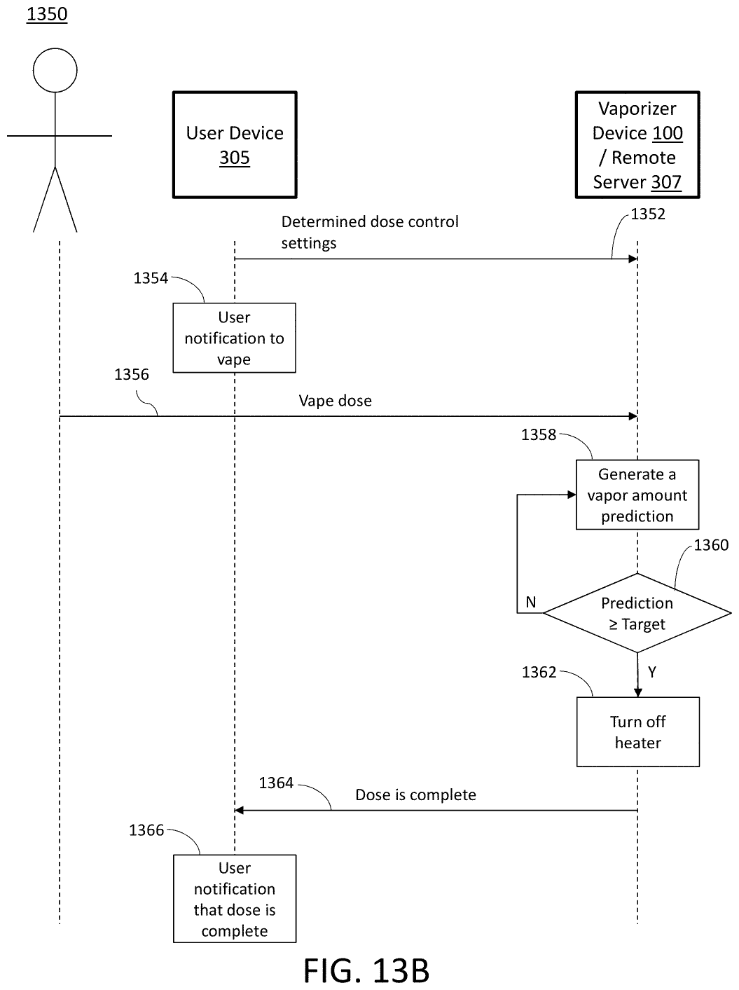

FIG. 13A is a swim lane diagram 1300 illustrating data exchange and communication consistent with implementations of the current subject matter. In particular, communication between the user device 305, the remote server 307, and the vaporizer device 100 is provided for implementing vapor production and dose control operations consistent with implementations of the current subject matter. In some implementations, the vaporizer device 100 may include some if not all of the user device 305 functionality.

At 1302 (“pair device with mobile app”), a request is sent to pair the user device 305 with the vaporizer device 100. In some instances, the pairing may be automatic once previously established. In some instances, each time the user connects to the app (e.g., the mobile app or web app), a pairing protocol with the vaporizer device 100 may be initiated. At 1304 (“device connected”), the vaporizer device 100 may send an acknowledgement message to the user device 305 indicating that the devices 305 and 100 are connected. In some instances, operations at 1302 and/or 1304 are not needed.

At 1306 (“select dose control”), the user may initiate or select one or more dose control options, such as dose size, via the app, and at 1308, the user device 305 may transmit the selected dose control options to the remote server 307. For example, in accordance with implementations of the current subject matter, various preset dose configurations may be established to control use of the vaporizer device 100 for the user. These preset dose configurations may be displayed to the user on a user interface of the user device 305 on which an application is running. The display of the preset dose configurations allows for the user to select a desired dose configuration. The preset dose configurations may relate to size, where size refers to size of the dose of vaporizable material, such as the amount of total particulate matter to be generated. According to some aspects, available dose sizes may include micro, small, medium, or large, signifying how much vapor is being produced for inhalation by a user. Other descriptive terms or identifiers (e.g., alphanumeric identifiers, symbols, and/or the like) may also be used for dose sizes, and the implementations described herein are not limited to the specific terms or identifiers describing the dose size. Consistent with implementations of the current subject matter, the desired dose may be a dose size with a fixed amount of total particulate matter, and the user may be able to select the quantity (e.g., one, two, three, four, or more) of doses.

Additionally, in accordance with implementations of the current subject matter, a setpoint temperature may be selected to be used for use of the vaporizer device 100. The setpoint temperature may be selected to, for example, adjust the strength of vapor being produced (for example, higher temperatures may produce a stronger vapor compared to vapor produced from a lower temperature). The selection of the setpoint temperature may in some instances affect the amount of vapor that is produced. The setpoint temperature may be based on a user selection via, for example, selection on the user device 305. Alternatively, a preset temperature (which may be user, system, or cartridge defined and may be based on various factors such as for example user preferences, crowdsourced information, type of vaporizable material, and/or the like) may be applied for use of the vaporizer device 100. If a user does not specify a desired setpoint temperature, the preset temperature may be a default setting (e.g., a default provided by the vaporizer device 100 or a default corresponding to the vaporizable material (e.g., a strain of cannabinoids)).

The trained model consistent with implementations of the current subject matter may be implemented at the remote server 307, such that when the remote server 307 receives the dose control options (at 1308 in the diagram 1300 (“selected dose control options”)), the dose control options are applied to the trained model to determine one or more operational settings to be applied by the vaporizer device 100. In particular, the dose control options may include a dose size, a dose amount, or a dose configuration that may be correlated with an amount of total particulate matter. By applying the dose size to the trained model trained to predict vapor production based on various operation metrics of the vaporizer device 100, the trained model uses the desired vapor production amount to determine one or more operation settings to be applied by the vaporizer device 100. For example, the trained model may determine an amount of energy to apply to the heater 166 during the user puff and/or the length of time for the heater 166 to remain on to achieve the desired vapor production amount.

According to some implementations, at 1310 (“determined dose control settings”), the remote server 307 may send to the user device 305 the one or more determined settings to be applied by the vaporizer device 100 to achieve the selected dose amount.

At 1312 (“determined dose control settings”), the user device 305 may send to the vaporizer device 100 the one or more determined settings to be applied to achieve the selected dose amount. In some implementations, the remote server 307 may send the one or more determined settings directly to the vaporizer device 100, rather than or in addition to sending the determined settings to the user device 305.

In response to receiving the one or more determined settings, the vaporizer device 100 applies the settings and sends, at 1314 (“ACK—dose control settings set”), an acknowledgement to the user device 305. The acknowledgement may indicate that the one or more determined settings are applied.

At 1316 (“user notification to vape”), the user device 305 may generate a user notification to indicate to the user that the user may begin using the vaporizer device 100 to consume the selected dose control option. The user notification may be generated, for example, on the user interface or through other feedback (e.g., haptics, sound, and/or the like). At 1318 (“vape dose”), the user may begin to use the vaporizer device 100.

FIG. 13B is a swim lane diagram 1350 illustrating data exchange and communication and operational aspects consistent with implementations of the current subject matter. In particular, the swim lane diagram 1350 illustrates aspects related to the vaporizer device 100 implementing vapor production and dose control operations consistent with implementations of the current subject matter. In some implementations, the vaporizer device 100 may include some if not all of the user device 305 functionality. The illustrated operations may occur after the dose control settings are determined by, for example, the remote server 307 and/or the vaporizer device 100 utilizing the trained model 560.

Consistent with implementations of the current subject matter, the trained model may compare TPM prediction to a target TPM. For example, sensor data may be sampled (e.g., at a repeated rate), and the trained model may use the sensor data to predict TPM. If the predicted TPM equals or exceeds the target TPM, power to the heater 166 may be terminated as the desired amount of TPM has been produced for consumption by the user.

At 1352, the determined dose control settings are provided by the user device 305 to the vaporizer device 100. In some implementations, the vaporizer device 100 determines the dose control settings, and 1352 is thus not needed. In some implementations, the determined dose control settings are provided by the remote server 307 to the vaporizer device 100. The determined dose control settings may correlate to a dose indicative of a desired amount of vapor or a target TPM, as selected or inputted by the user. In response to receiving the one or more determined dose control settings, the vaporizer device 100 applies the settings and may send an acknowledgement to the user device 305. The acknowledgement may indicate that the one or more determined settings are applied.

At 1354 (“user notification to vape”), the user device 305 may generate a user notification to indicate to the user that the user may begin using the vaporizer device 100 to consume the selected dose control option. The user notification may be generated, for example, on the user interface or through other feedback (e.g., haptics, sound, and/or the like). At 1356 (“vape dose”), the user may begin to use the vaporizer device 100 to consume the dose.

At 1358, a vapor amount prediction is generated by the vaporizer device 100. For example, and consistent with implementations of the current subject matter, the vaporizer device 100 processes at least one operation metric with the trained model 560 to generate a vapor amount or TPM prediction. The at least one operation metric may be collected from the vaporizer device 100 during the use of the vaporizer device 100, and/or may be sensor data sampled at, for example, a repeated rate. The trained model 560 may use the operation metrics and/or the sensor data to predict the amount of vapor or TPM.

At 1360, the vaporizer device 100 compares the predicted amount of vapor against a target amount, which may be the dose or associated with the dose selected by the user or otherwise inputted. If, as shown at 1362, the predicted amount of vapor (as determined by the trained model 560) equals or exceeds the target amount of vapor (as inputted or selected by the user), power to the heater 166 is terminated. As the desired amount of vapor has been produced for consumption by the user, the vaporizer device 100 responds by turning off (e.g., terminating power to the heater 166). If the predicted amount of vapor does not equal or exceed the target amount of vapor, the vaporizer device 100 may generate a new prediction based on updated operation metrics and/or sensor data. According to aspects of the subject matter, the new prediction may be generated after a predetermined period of time has elapsed. For example, consistent with implementations of the current subject matter, a user may select a dose and begin using the vaporizer device 100. The vaporizer device 100 may generate at a regular interval (e.g., after the predetermined period of time has elapsed) the prediction using the trained model 560. Once the predicted amount of vapor equals or exceeds the target amount of vapor, the vaporizer device 100 stops functioning (e.g., for at least a period of time until a subsequent use or another dose is selected).

At 1364, the vaporizer device 100 sends to the user device 305 a notification that the dose is complete. At 1366, the user device 305 generates a user notification indicating that the dose is complete. In some implementations, the user device 305 does not generate a notification, and the user is made aware of completion of the dose by the vaporizer device 100 not further responding (e.g., until a subsequent use).

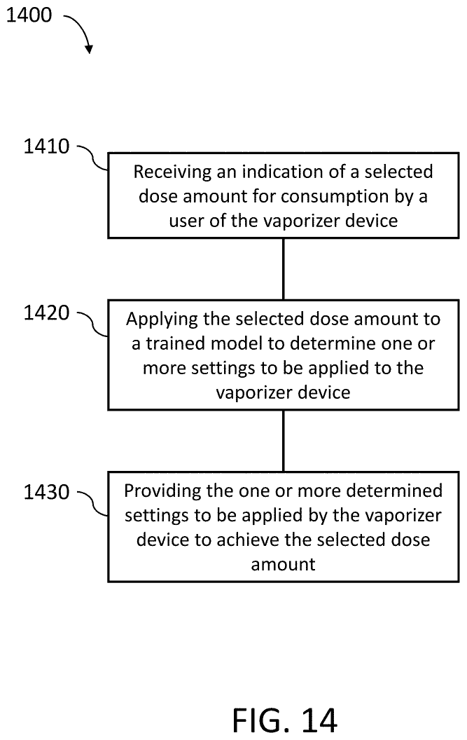

With reference to FIG. 14, a process chart 1400 illustrates features of a method, which may optionally include some or all of the following.

At 1410, an indication of a selected dose amount of a vaporizable material for consumption by the user of the vaporizer device 100 may be received. The remote server 307 may receive the indication from the user device 305 associated with the vaporizer device 100, or the user device 305 may receive and use the indication. For example, as described herein, the user may initiate or select one or more dose control options, such as dose size, via the app, and the user device 305 may transmit the selected dose control options to the remote server 307.

At 1420, the selected dose amount may be applied to a trained model to determine one or more settings to be applied by the vaporizer device 100 to achieve the selected dose amount. In particular, the dose amount may be correlated with an amount of total particulate matter. By applying the dose amount to the trained model trained to predict vapor production based on various operation metrics of the vaporizer device 100, the trained model may use the desired vapor production amount to determine one or more operational settings to be applied by the vaporizer device 100. For example, the model may determine an amount of energy to apply to the heater 166 during the user puff.

At 1430, the one or more determined settings are provided to be applied by the vaporizer device 100 to achieve the selected dose amount. For example, the remote server 307 may send to the user device 305 the one or more determined settings to be applied by the vaporizer device 100. The user device 305 may, in turn, send the one or more determined settings to the vaporizer device 100, which applies the settings and sends a corresponding acknowledgement to the user device 305. As another example, the user device 305 may determine the one or more settings by implementing the trained model, and may provide the one or more determined settings to the vaporizer device. As another example, in some instances, the vaporizer device 100 may receive the selected dose amount, determine the one or more settings, and apply the one or more determined settings. The user device 305 and/or the vaporizer device 100 may, in some instances, generate an indication to the user that the appropriate settings are applied to achieve the selected dose amount, and that user may begin use of the vaporizer device 100.

In some implementations, a temperature selection may be applied with the selected dose amount to the trained model to determine the one or more settings to be applied by the vaporizer device. That is, temperature may be an additional input into the trained model.

According to additional aspects of the current subject matter, the trained 560 model may also be used to determine usage data related to the cartridge 150. For example, a particular cartridge 150 may have associated with it an initial mass (e.g., about 500 mg). At the end of each user puff, collected data related to the puff (e.g., the use of the vaporizer device 100 and/or the cartridge 150) may be applied to the trained model 560 to estimate the amount of vapor produced in, for example, milligrams of total particulate matter. As the initial mass is known, the predicted total particulate matter may be subtracted from the initial mass to represent an estimated amount of remaining vaporizable material in the cartridge 150. This may serve as a type of fuel gauge for the user to be informed of amount of vapor used and amount of vapor remaining, which may be displayed (e.g., graphically) to the user for usage tracking.

In another implementation, the cartridge 150 may be associated with a total amount of total particulate matter. During use of the vaporizer device 100, the trained model 560 may determine (e.g., for each puff) the amount of vapor produced in terms of total particulate matter. This may allow for determination (e.g., by the remote server 307, the vaporizer device 100, and/or the user device 305) of an amount of total particulate matter used and an amount of total particulate matter remaining.

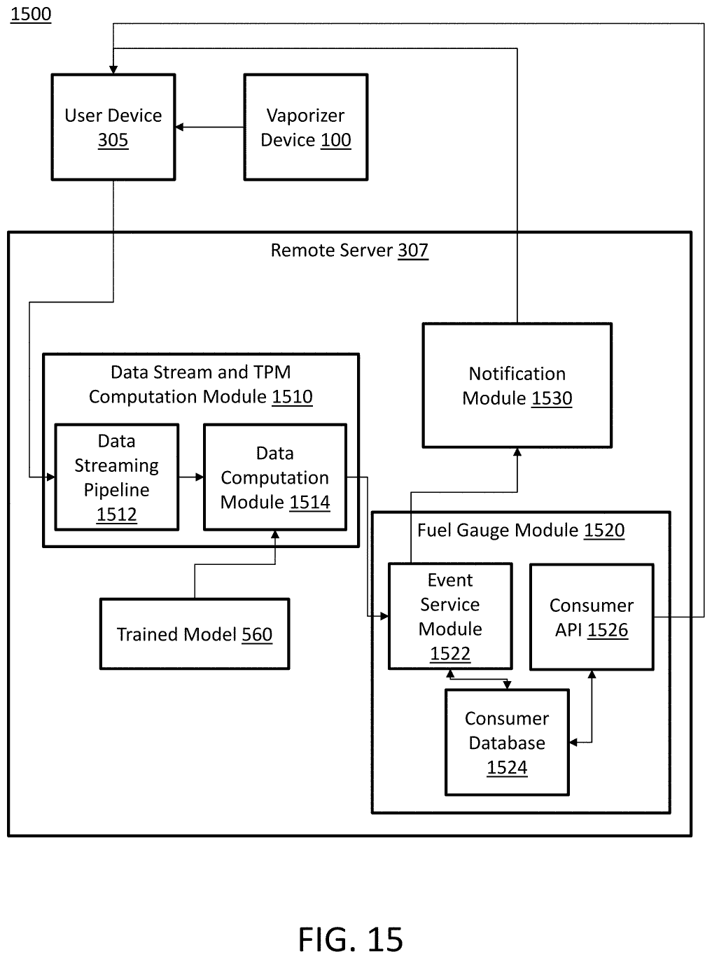

With reference to FIG. 15, a block diagram 1500 illustrates aspects related to a fuel gauge implementation consistent with implementations of the current subject matter. The remote server 307 is connected to the user device 305, on which a web-based application and/or a mobile application may be executing, as described herein (e.g., at FIG. 3). The remote server 307, incorporating the trained model 560 and other modules or services described herein, provides for monitoring an amount of vapor produced by the vaporizer device 100 connected to the user device 305 and providing to a user an indication of a remaining amount of vapor.

According to aspects of the current subject matter, the user device 305 pulls logs (e.g., telemetry logs) related to operation from the vaporizer device 100. For example, sensor data and other parameters during operation of the vaporizer device 100 are collected by the user device 305. The sensor data and other parameters may be collected for each puff taken by a user or at other defined intervals.

The logs pulled by the user device 305 are provided to the data stream and TPM computation module 1510. In particular, a data streaming pipeline 1512, such as Kinesis Stream and/or Kinesis Firehose, receives the logs and provides the logs for processing to a data computation module 1514, such as a lambda function. According to aspects of the current subject matter, the data computation module 1514 selects the data from the logs that is associated with a user puff on the cartridge 150 of the vaporizer device 100. For example, the data computation module 1514 may extract the following fields for use as inputs to the trained model 560 along with a cartridge identifier of the cartridge 150 and a user identifier associated with the vaporizer device 100 and/or the cartridge 150: a puff number; an air path pressure; an ambient temperature; a maximum differential pressure; a differential pressure integral (e.g., the integral of the pressure differential between the air path and ambient pressures); a sum squared error after reaching a setpoint temperature (e.g., the sum of the squared differences between setpoint temperature and heater temperature after a lower bound of the setpoint temperature has been crossed); an energy amount used at reaching a setpoint temperature; an energy amount used during a puff; time to reach a setpoint temperature; length of a puff; a baseline resistance value of the heater 166; a temperature of the heater 166 at a start of a puff; a temperature of the heater 166 at the end of a puff; a resistance value of the heater 166 at the end of a puff; a voltage of the battery 124 at a start of a puff; a voltage of the battery 124 at the end of a puff; a minimum recorded voltage of the battery 124; a resistance of the heater 166 at a setpoint temperature; and/or a setpoint temperature of the heater 166.

The data computation module 1514 may communicate with the trained model 560 to compute an amount of vapor consumed by the user during the puff, consistent with implementations of the current subject matter. For example, the data computation module 1514 may load a current version of the trained model 560 for use with the streamed puff data to compute the amount of vapor, or TPM, consumed for the particular cartridge 150 associated with the cartridge identifier.

A fuel gauge module 1520 may include one or more modules, components, and/or services for receiving the computed TPM, associating the computed TPM for the particular cartridge 150 with the user, and determining or identifying the amount of TPM consumed for the particular cartridge 150 and the amount of TPM remaining. The fuel gauge module 1520 may also interface with a notification module 1530 to communicate the amount of TPM consumed and/or remaining. In some implementations, the fuel gauge module 1520 may interface with the user device 305.

In particular, an event service module 1522 receives the TPM computed by the trained model 560 from the data computation module 1514, and stores the computed TPM for the particular cartridge 150 in a consumer database 1524. At the consumer database 1524, a cartridge identifier associated with the cartridge 150 is also associated with a user identifier associated with a user. Data related to one or more user devices of the user is associated with the user identifier. One or more fields in the consumer database 1524 may be updated with the computed TPM for the cartridge 150 to track overall TPM production for the cartridge 150. For example, each time the TPM is computed, the consumer database 1524 may be updated with the new TPM computation so that the consumer database 1524 is tracking and/or updating TPM production on a, for example, per puff basis. The consumer database 1524 also includes an initial, total amount of TPM of the cartridge 150 so that an amount of vapor or TPM remaining in the cartridge 150 may be determined. A consumer application program interface (API) 1526 pulls, from the consumer database 1524, a total amount of TPM consumed and/or an amount of TPM remaining in the cartridge 150, as well as a user's device or devices associated with the cartridge identifier.

Consistent with implementations of the current subject matter, the event service module 1522 may provide the notification module 1530 a notification when the amount of TPM remaining in the cartridge 150 falls below a threshold. The threshold may be defined by the user who wishes to know when there is 75% remaining, 50% remaining, 25% remaining, 10% remaining, 5% remaining, and/or other defined threshold. The threshold may be associated with the cartridge 150 and/or the vaporizer device 100, or the threshold may be system defined. In some implementations, the threshold may be user-defined and/or user-adjustable. In some implementations, rather than or in addition to a threshold, the notification may be provided after each puff or other defined metric such as at a periodic interval or after an amount of time has elapsed. Notification settings, such as the threshold and the defined metric, may be user-definable and/or user-adjustable and/or may be associated with the cartridge 150 and/or the vaporizer device 100. The notification generated by the consumer API 1526 may include the notification settings and the user's device or devices, which are defined to receive the notification.

The user device 305 may fetch from the consumer API 1526 a total amount of TPM consumed and/or an amount of TPM remaining in the cartridge 150 associated with the user of the user device 305. The fetching may be performed, for example, in a periodic pulling fashion. This allows for the user device 305 to be updated with the TPM consumption and TPM remaining information for presentation on a display of the user device 305.

In some implementations, the notification module 1530, upon receiving the notification and settings and the user's device or devices from the event service module 1522, may send the notification to the user's device or devices. The notification module 1530 may include, for example, a push notification service and/or an email notification service.

Aspects of the current subject matter advantageously provide a model, such as a machine learning model and/or a statistical model, that predicts an amount of vapor production of a vaporizable material by a vaporizer device, the amount of vapor production being a function of various factors relating to operation of the vaporizer device. Aspects of the current subject matter also relate to integration of the model into a system to provide user control and visibility into the vapor production. The model and integration thereof provides users with a consistent, predictable, and reliable experience.

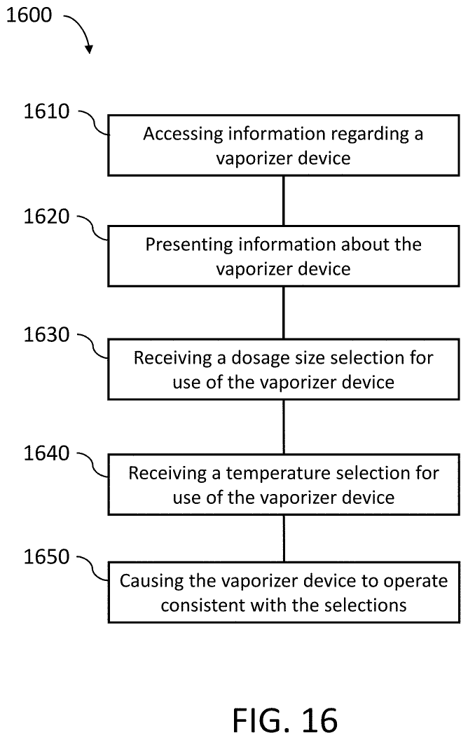

With reference to FIG. 16, a process chart 1600 illustrates features of a method, which may optionally include some or all of the following.

At 1610, information regarding a vaporizer device is accessed through operation of an application executing on one or more programmable processors. For example, through use of an app running on a user device 305 and connected to a vaporizer device 100, features of the vaporizer device 100 and a cartridge 150 may be accessed. Such features may include, for example, the type of vaporizable material in the cartridge 150, associated properties, and historical usage information.

At 1620, information about the vaporizer device 100 is presented using a user interface generated on a display by the one or more processors. For example, information about the vaporizer device 100, the cartridge 150, and/or the vaporizable material may be displayed on the user device 305. Additionally, optional dose sizes and temperature options may be presented, allowing the user to select one or more parameters for use of the vaporizer device 100. For example, preset dose configurations may be displayed to the user on a user interface, allowing for the user to select a desired dose. The preset dose configurations may relate to dose size, where dose size refers to size or amount of vaporizable material.

At 1630, a dosage size selection is received. The dosage size selection may be provided by user input with the user interface and may include a dose size as described here. For example, according to some aspects, available dose sizes may include micro, small, medium, or large, signifying how much vapor is being produced for inhalation by a user. Other descriptive terms or identifiers (e.g., symbols or the like) may also be used for dose sizes.

At 1640, a temperature selection indicating a temperature for use with the vaporizer device 100 is received. The setpoint temperature may be selected to, for example, adjust the strength of vapor being produced (for example, higher temperatures may produce a stronger vapor compared to vapor produced from a lower temperature). The selection of the setpoint temperature may in some instances affect the amount of vapor that is produced. The setpoint temperature may be based on a user selection via, for example, selection on the user device 305. Alternatively, a preset temperature may be applied for use of the vaporizer device 100.

At 1650, the vaporizer device 100 is caused to operate consistent with the dosage size selection and the temperature selection. For example, the vaporizer device 100 operates at the selected setpoint temperature to produce the selected dosage size. Consistent with implementations of the current subject matter, the trained model 560 is utilized to determine when the selected dosage amount has been generated.

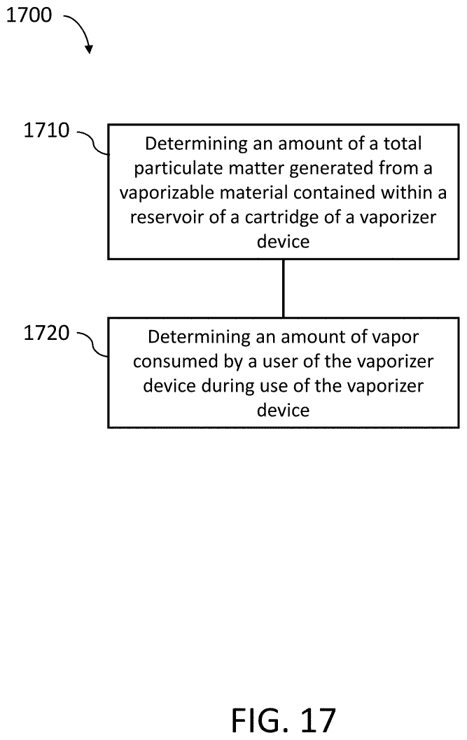

With reference to FIG. 17, a process chart 1700 illustrates features of a method, which may optionally include some or all of the following.

At 1710, a controller 128 of a vaporizer device 100 determines an amount of total particulate matter generated from a vaporizable material contained within a reservoir of a cartridge 150 of the vaporizer device 100 during use of the vaporizer device 100. The amount of total particulate matter may be correlated with an amount of energy supplied to heat the vaporizable material. For example, there may be a linear correlation between the total particulate matter and the amount of energy. The amount of total particulate matter generated may also be based on characteristics of the use of the vaporizer device 100 (e.g., number of puffs, length of time per puff, etc.), properties of the vaporizable material, information related to ambient conditions, and characteristics of a heater contained within the cartridge and configured to heat the vaporizable material (e.g., properties and/or materials of a wick, and/or properties and/or materials of a heating element).

At 1720, the controller 128 determines an amount of vapor consumed by a user of the vaporizer device 100 during a particular use or over a lifetime of the cartridge 150. The vapor is produced from heating of the vaporizable material, and the amount of vapor consumed is a function of the total particulate matter generated. The amount of vapor consumed may further be a function of potency characteristics of the vaporizable material (e.g., the chemical composition and age of the vaporizable material), a temperature at which the vaporizable material is heated, a particle size distribution of the vapor, information related to energy consumption, properties of the vaporizable material, usage history of the cartridge, ambient conditions, and characteristics of a heater (including for example a wick and heating element) contained within the cartridge and configured to heat the vaporizable material.

In some examples, the vaporizable material may include a viscous liquid such as, for example a cannabis oil. In some variations, the cannabis oil comprises between 0.3% and 100% cannabis oil extract. The viscous oil may include a carrier for improving vapor formation, such as, for example, propylene glycol, glycerol, medium chain triglycerides (MCT) including lauric acid, capric acid, caprylic acid, caproic acid, etc., at between 0.01% and 25% (e.g., between 0. 1% and 22%, between 1% and 20%, between 1% and 15%, and/or the like). In some variations the vapor-forming carrier is 1,3-Propanediol. A cannabis oil may include a cannabinoid or cannabinoids (natural and/or synthetic), and/or a terpene or terpenes derived from organic materials such as for example fruits and flowers. For example, any of the vaporizable materials described herein may include one or more (e.g., a mixture of) cannabinoid including one or more of: CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), Tetrahydrocannabinol, Cannabidiol (CBD), Cannabinol (CBN), Tetrahydrocannabinolic Acid (THCA), Cannabidioloc Acid (CBDA), Tetrahydrocannabivarinic Acid (THCVA), one or more Endocannabinoids (e.g., anandamide, 2-Arachidonoylglycerol, 2-Arachidonyl glyceryl ether, N-Arachidonoyl dopamine, Virodhamine, Lysophosphatidylinositol), and/or a synthetic cannabinoids such as, for example, one or more of: JWH-018, JWH-073, CP-55940, Dimethylheptylpyran, HU-210, HU-331, SR144528, WIN 55,212-2, JWH-133, Levonantradol (Nantrodolum), and AM-2201. The oil vaporization material may include one or more terpene, such as, for example, Hemiterpenes, Monoterpenes (e.g., geraniol, terpineol, limonene, myrcene, linalool, pinene, Iridoids), Sesquiterpenes (e.g., humulene, farnesenes, farnesol), Diterpenes (e.g., cafestol, kahweol, cembrene and taxadiene), Sesterterpenes, (e.g., geranylfarnesol), Triterpenes (e.g., squalene), Sesquarterpenes (e.g, ferrugicadiol and tetraprenylcurcumene), Tetraterpenes (lycopene, gamma-carotene, alpha- and beta-carotenes), Polyterpenes, and Norisoprenoids. For example, an oil vaporization material as described herein may include between 0.3-100% cannabinoids (e.g., 0.5-98%, 10-95%, 20-92%, 30-90%, 40-80%, 50-75%, 60-80%, etc.), 0-40% terpenes (e.g., 1-30%, 10-30%, 10-20%, etc.), and 0-25% carrier (e.g., medium chain triglycerides (MCT)).

In any of the oil vaporizable materials described herein (including in particular, the cannabinoid-based vaporizable materials), the viscosity may be within a predetermined range. The range may be between, at room temperature (23° C.) about 30 cP (centipoise) and 115 kcP (kilocentipoise), between 30 cP and 200 kcP, although higher viscosities and/or lower viscosities may be implemented as well. For example, the viscosity may be between 40 cP and 113 kcP at room temperature. Outside of this range, the vaporizable material may fail in some instances to wick appropriately to form a vapor as described herein. In particular, it is typically desired that the oil may be made sufficiently thin to both permit wicking at a rate that is useful with the apparatuses described herein, while also limiting leaking (e.g., viscosities below that of ˜30 cP at room temperature might result in problems with leaking).

Although the disclosure, including the figures, described herein may described and/or exemplify these different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A system, comprising:
at least one data processor; and
at least one memory storing instructions which, when executed by the at least one data processor, cause operations comprising:
receiving, at a trained model, at least one metric of a vaporizer device during a user puff on the vaporizer device; and
processing, at the trained model, the at least one metric to predict an amount of vapor production of a vaporizable material from the vaporizer device during the user puff on the vaporizer device.

2. The system of claim 1, wherein the operations further comprise:
training a model to provide the trained model configured to predict the amount of vapor production of the vaporizable material from the vaporizer device during the user puff on the vaporizer device.

3. The system of claim 2, wherein the training of the model comprises processing, with the model, training data, the training data comprising a plurality of metrics corresponding to operation of the vaporizer device.

4. The system of claim 3, wherein the training data comprises a plurality of values from a plurality of puffs on the vaporizer device.

5. The system of claim 2, wherein the model comprises a machine learning model, a statistical model, and/or an analytical model.

6. The system of claim 5, wherein the model comprises one of a regression model, a random forest model, an extra trees regression model, an adaptive boosted random forest regression model, an adaptive boosted extra trees regression model, a gradient boosting regression model, a support vector regression, a linear regression model, a ridge regression model, an elastic net regression model, and a lasso regression model, or a k-nearest neighbors regression model.

7. The system of claim 6, wherein the model comprises the regression model, and wherein the regression model averages predictions of regression trees, the regression trees individually built on a random subset of the training data and a random subset of variables.

8. The system of claim 1, wherein the at least one metric comprises one or more of the following: a metric obtained during operation of the vaporizer device, an amount of energy used during the user puff, a temperature of a heating element of the vaporizer device, a differential pressure value of the vaporizer device, a resistance value of the heating element of the vaporizer device, a battery voltage of a battery of the vaporizer device, an amount of time to reach a setpoint temperature of the vaporizer device, an ambient pressure value, an amount of time of the user puff, an amount of energy to reach the setpoint temperature, an air path pressure in an air path of the vaporizer device, an ambient temperature, and an air path temperature in the air path of the vaporizer device.

9. The system of claim 1, wherein the operations further comprise:
determining, based on the predicted amount of vapor production and an initial, total amount of vapor associated with a cartridge of the vaporizer device, a remaining amount of vapor; and
providing, upon a determination that the remaining amount of vapor is less than a predefined threshold, a notification to a user device associated with the vaporizer device.

10. The system of claim 9, wherein the operations further comprise:
storing, at a database, the predicted amount of vapor production with a cartridge identifier associated with the cartridge and a user identifier associated with a user.

11. The system of claim 1, wherein the operations further comprise:
receiving, an indication of a desired amount of vapor production;
determining that the desired amount of vapor production equals or exceeds the predicted amount of vapor production; and
causing, in response to the determination that the desired amount of vapor production equals or exceeds the predicted amount of vapor production, the vaporizer device to stop producing vapor.

12. The system of claim 11, wherein the predicted amount of vapor production is summed over a plurality of user puffs.

13. A method, comprising:
receiving, at a trained model, at least one metric of a vaporizer device during a user puff on the vaporizer device; and
processing, at the trained model, the at least one metric to predict an amount of vapor production of a vaporizable material from the vaporizer device during the user puff on the vaporizer device.

14. The method of claim 13, further comprising:
training a model to provide the trained model configured to predict the amount of vapor production of the vaporizable material from the vaporizer device during the user puff on the vaporizer device.

15. The method of claim 14, wherein the training of the model comprises processing, with the model, training data, the training data comprising a plurality of metrics corresponding to operation of the vaporizer device.

16. The method of claim 15, wherein the training data comprises a plurality of values from a plurality of puffs on the vaporizer device.

17. The method of claim 14, wherein the model comprises a machine learning model, a statistical model, and/or an analytical model.

18. The method of claim 17, wherein the model comprises one of a regression model, a random forest model, an extra trees regression model, an adaptive boosted random forest regression model, an adaptive boosted extra trees regression model, a gradient boosting regression model, a support vector regression, a linear regression model, a ridge regression model, an elastic net regression model, and a lasso regression model, or a k-nearest neighbors regression model.

19. The method of claim 18, wherein the model comprises the regression model, and wherein the regression model averages predictions of regression trees, the regression trees individually built on a random subset of the training data and a random subset of variables.

20. The method of claim 13, wherein the at least one metric comprises one or more of the following: a metric obtained during operation of the vaporizer device, an amount of energy used during the user puff, a temperature of a heating element of the vaporizer device, a differential pressure value of the vaporizer device, a resistance value of the heating element of the vaporizer device, a battery voltage of a battery of the vaporizer device, an amount of time to reach a setpoint temperature of the vaporizer device, an ambient pressure value, an amount of time of the user puff, an amount of energy to reach the setpoint temperature, an air path pressure in an air path of the vaporizer device, an ambient temperature, and an air path temperature in the air path of the vaporizer device.

21. The method of claim 13, further comprising:
determining, based on the predicted amount of vapor production and an initial, total amount of vapor associated with a cartridge of the vaporizer device, a remaining amount of vapor; and
providing, upon a determination that the remaining amount of vapor is less than a predefined threshold, a notification to a user device associated with the vaporizer device.

22. The method of claim 21, further comprising:
storing, at a database, the predicted amount of vapor production with a cartridge identifier associated with the cartridge and a user identifier associated with a user.

23. The method of claim 13, further comprising:
receiving, an indication of a desired amount of vapor production;
determining that the desired amount of vapor production equals or exceeds the predicted amount of vapor production; and
causing, in response to the determination that the desired amount of vapor production equals or exceeds the predicted amount of vapor production, the vaporizer device to stop producing vapor.

24. The method of claim 23, wherein the predicted amount of vapor production is summed over a plurality of user puffs.

25. A non-transitory computer readable medium storing instructions, which when executed by at least one data processor, result in operations comprising:
receiving, at a trained model, at least one metric of a vaporizer device during a user puff on the vaporizer device; and
processing, at the trained model, the at least one metric to predict an amount of vapor production of a vaporizable material from the vaporizer device during the user puff on the vaporizer device.

26. A system, comprising:
at least one data processor; and
at least one memory storing instructions which, when executed by the at least one data processor, cause operations comprising:
receiving an indication of a selected dose amount of a vaporizable material for consumption by a user of a vaporizer device;
providing the selected dose amount to a trained model to determine one or more settings to be applied by the vaporizer device;
applying the one or more settings to the vaporizer device to achieve the selected dose amount.

27. The system of claim 26, wherein the selected dose amount correlates with an amount of total particulate matter of the vaporizable material.

28. The system of claim 26, wherein the one or more settings comprise an amount of energy to apply to a heating element of the vaporizer device.

29. The system of claim 26, further comprising:
receiving a temperature selection; and
providing the temperature selection with the selected dose amount to the trained model to determine the one or more settings to be applied by the vaporizer device.

30. The system of claim 26, wherein the trained model is trained to predict an amount of vapor production of the vaporizable material from the vaporizer device during a puff on the vaporizer device.

31. The system of claim 30, wherein the trained model is trained based at least on training data, the training data comprising a plurality of metrics corresponding to operation of the vaporizer device.

32. The system of claim 26, wherein the trained model comprises a machine learning model, a statistical model, and/or an analytical model.
